(19) United States
(12) Patent Application Publication
(10) Pub. No.: US 2008/0226566 A1
(43) Pub. Date: Sep. 18, 2008

(54) USE OF POORLY WATER-SOLUBLE CALCIUM SALTS AND/OR THE COMPOSITES THEREOF

(76) Inventors: Tilo Poth, Weinheim (DE); Jorg Peschke, Gross-Zimmern (DE); Holger Franke, Ginsheim-Gustavsburg (DE); Rainer Kliss, Reinheim-Zeilhard (DE); Heidi Schweitzer, Pfungstadt (DE)

Correspondence Address: PAUL & PAUL, 2000 MARKET STREET, PHILADELPHIA, PA, 19103-3229, US

(21) Appl. No.: 12/105,149

(22) Filed: Apr. 17, 2008

Related U.S. Application Data
(63) Continuation of application No. PCT/EP06/10240, filed on Oct. 24, 2006.

(30) Foreign Application Priority Data
Oct. 31, 2005 (DE) 10 2005 052 371.4
Mar. 1, 2006 (DE) 10 2006 009 793.9

(51) Int. Cl. A61K 8/21; A61K 8/19; A61Q 11/00

(52) U.S. Cl. 424/52; 424/53

(57) ABSTRACT

The use of a composition containing at least one not easily water-soluble calcium salt and/or the composite material thereof, to protect and/or therapeutically treat and/or preventively treat teeth and/or bones in case of damage or prevent damage resulting from external influences, especially biological, chemical, physical, and/or microbiological influences, particularly to prevent and repair bone and tooth erosion, especially the enamel, maintain the enamel, protect teeth from aggressive acids, particularly caused by bacterial activity or the effect of acids contained in food, protect teeth from demineralizing, seal cracks, provide protection against and/or repair primary lesions and/or initial cavities in the enamel, smooth the tooth surface, prevent cavities make it easier to clean teeth, improve the mechanical resistance of teeth, and generally keep teeth healthy.

### CROSS-REFERENCE TO RELATED APPLICATIONS

This application is a continuation under 35 U.S.C. Section 365(c) and 35 U.S.C. Section 120 of International Application No. PCT/EP2006/010240, filed Oct. 24, 2006. This application also claims priority under 35 U.S.C. Section 119 of German Patent Applications No. 10 2006 009 793.9, filed Mar. 1, 2006, and No. 10 2005 052 371.4, filed Oct. 31, 2005.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to the use of a composition comprising at least one poorly water-soluble calcium salt and/or a composite material thereof for the protection and/or for the therapeutic and/or preventive treatment of teeth and/or bones in the case of or against damage which can be attributed to external influences, in particular, of body-related, chemical, physical and/or microbiological type, in particular, for prevention and repair of erosion of bones and teeth, in particular, enamel, care of the enamel, and for protection of the teeth against attack by acids, in particular, due to bacterial activity or due to action of acids from foods, for protection against demineralization of the teeth, for sealing of fissures, for protection against and/or repair of primary lesions and/or initial caries in the enamel and for smoothing the surface of the tooth, for prevention of caries, for improving the cleanability and the mechanical resistance of teeth, and dental health generally.

Phosphate salts of calcium have been added to the formulations of dentifrices and dental care compositions for a long time as abrasive components and for promoting remineralization of the enamel. This applies, in particular, to hydroxyapatite and fluoroapatite, and to amorphous calcium phosphates and to brushite (dicalcium phosphate dihydrate). Calcium fluoride has also repeatedly been described as a constituent of dentifrices, as a component for strengthening the enamel, and for caries prophylaxis.

The availability of calcium compounds for the desired remineralization depends very crucially on the particle size of these components, which are poorly water-soluble and dispersed in the dental care compositions. It has, therefore, been proposed to employ these poorly soluble calcium salts in very fine dispersion.

The enamel and the supportive tissue of the bones consist mainly of the mineral hydroxyapatite. In the biological formation process, hydroxyapatite adds in an ordered manner to the protein matrix in the bone or tooth, which consists mainly of collagen. The formation of the hard and loadable mineral structures is controlled here by the “matrix proteins” which, in addition to collagen, are formed by further proteins which add to the collagen and thus bring about a structured mineralization process, which is also called “biomineralization.”

The enamel, which lies over the dentine and covers the crown of the tooth in a varying thickness, is particularly hard and is the most important protective shield of the tooth. In the moist oral cavity, it is in a continuous de- and remineralization equilibrium with the saliva, which is rich in minerals. This enamel is, therefore, able to compensate for mineral losses in the dental material, again under ideal conditions.

It is known that acids in the oral cavity, e.g., due to the consumption of acidic or acid-containing foods, for example, fruit juices, lemonades, yoghurt, salad dressing, fruit or wine, can soften and finally dissolve the enamel. Even after exposure times which are customary for consumption, a marked change in the teeth can be detected, for example, by means of an increase in the surface roughness. Acids absorbed via the food are also designated as extrinsic.

Endogenous, intrinsic acids, and various diseases, can damage the dental material. Thus, gastric acid reaches the oral cavity owing to eructation, regurgitation, heartburn or due to vomiting, and has a particularly highly damaging effect because of its low pH. The same also applies in connection with pathological eating disorders, such as, in particular, bulimia. All of these processes interfere with the natural equilibrium between the mineral absorption and attrition in the teeth so strongly that the natural therapeutic capacity of the saliva is no longer able to bring about restoration of the hard tissue. The result is an irreversible loss of dental material.

In various forms of diminished saliva flow (hyposalivation) or dryness of the mouth (xerostomatia) too, the regeneration of the dental material mentioned is no longer naturally possible due to too low a flow of saliva.

If the natural regeneration of the dental material fails, a softer enamel increasingly results, which is no longer able to fulfill its natural protective function. It is much more vulnerable to mechanical stress than healthy, solid enamel.

Even simple toothbrushing does not lead to an improvement here. On the other hand, it was found that toothbrushing leads directly or with a delay of some time after the consumption of acid-containing foods, to abrasion of the softened enamel. In addition, toothbrushing with strongly abrasive-containing toothpastes also accelerates the loss of dental substance.

Furthermore, the damage to the enamel favors the colonization of the tooth with bacteria. The bacteria produce further acids due to metabolic processes in very close proximity to the enamel, which can result in caries.

(2) Description of Related Art, Including Information Disclosed Under 37 C.F.R. Sections 1.97 and 1.98.

“Bone substitutes,” which promote the natural biomineralization process, play an important role in the restoration of bone material. Such agents are also needed for the coating of implants, in order to achieve substance-to-substance connections between bone and implants, by which tensile forces can also be transmitted. Of particular importance here are coatings with a high bioactivity, which lead to effective composite osteogenesis. According to the prior art, as is described, for example, by B. G. Willmann in Mat.-Wiss. u. Werkstofftech. 30 (1999), 317, hydroxyapatite is generally applied to implants. In addition to the often inadequate acceleration of the biomineralization process, the breaking off of the hydroxyapatite layers and their unsatisfactory chemical stability is disadvantageous in this procedure.

For certain applications, bone substitute materials applicable in liquid form are needed. A particularly small particle size is necessary here, which, however, cannot be achieved satisfactorily with the conventional bone substitutes. Above and beyond the disadvantages of the application technology (lack of dispersibility of the solid constituents), the previously available bone substitute materials which can be applied in liquid form at best exhibit a biocompatible, possibly resorbable action because of the coarsely crystalline inorganic components and the lack of bioanalogous organic components. However, osteoinductive, osteoconductive and osteostimulating materials directly promoting natural biomineralization and thus, also bone growth are desired.

Among the bone substitutes, composites of hydroxyapatite and collagen are of particular interest, since they imitate the composition of natural bone. A similar situation prevails in the restoration of dental material: dentine consists of approximately 30 percent protein (essentially collagen) and up to 70 percent mineral substances (essentially hydroxyapatite). Enamel, on the other hand, consists of approximately 95 percent hydroxyapatite and approximately 5 percent proteins.

Composite materials of the type described are accessible synthetically, as described, for example, by B. Flautre et al. in J. Mater. Sci.: Mater. In Medicine 7 (1996), 63. However, in these composites the particle size of the calcium salts is over 1,000 nm, which is too coarse to achieve a satisfactory biological action as a remineralizing agent.

On the other hand, R. Z. Wang et al., J. Mater. Sci. Lett. 14 (1995), 490 describe a production process for a composite material made of hydroxyapatite and collagen, in which hydroxyapatite is deposited on the collagen matrix in uniformly dispersed form with a particle size in the range from 2 to 10 nm. The composite material should have a better biological activity compared to other hydroxyapatite/collagen composites known from the prior art because of the finely divided nature of the hydroxyapatite. As described below, however, the composite material described by R. Z. Wang et al. also does not adequately fulfill the need for composite materials which imitate the composition and the microstructure of natural bone and tooth material and are suitable in a completely satisfactory manner for the remineralization of these natural materials.

In EP 1 139 995 A1, it is proposed to stabilize suspensions of calcium salts which are poorly soluble in water in very finely divided form during precipitation, or shortly thereafter by carrying out the precipitation in the presence of an agglomeration inhibitor or redispersing the dispersion in the presence of the agglomeration inhibitor, e.g., a protective colloid or surfactant.

WO 01/01930 discloses composite materials which are constructed from nanoparticulate poorly soluble calcium salts and protein components and which have a remineralizing action on enamel and dentine.

A further disadvantage of protein-containing composite materials known from the prior art consists in their often laborious production. For instance, in the production of the composite of hydroxyapatite and collagen described in R. Z. Wang et al. insoluble collagen must be handled and dispersed in very large amounts of solvent, which is technically laborious. This process additionally raises problems with respect to the disposal of the waste water formed during production.

### BRIEF SUMMARY OF THE INVENTION

It has now been found that poorly water-soluble calcium salts and/or their composite materials, in addition to the re- and neomineralization of dental material, are also able to restore the attacked surfaces on bones and teeth, in particular, attacked enamel, and thus to protect the teeth, inter alia, against erosion and caries. Overall, an improvement in dental health is thus generally brought about.

### BRIEF DESCRIPTION OF THE SEVERAL VIEWS OF THE DRAWING(S)

FIG. 1: Integrated mineral loss.

Baseline (untreated sample), placebo (use of the gel formulation without active substance), fluoride (use of the gel formulation containing 900 ppm of fluoride), apatite-protein composite+900 ppm of fluoride), apatite-protein composite+900 ppm fluoride (use of the gel formulation containing 1% of apatite-protein composite and 900 ppm of fluoride).

FIG. 2: Microhardness after treatment of acid-damaged enamel disks of Groups 1 and 2 with toothpaste suspensions (in percent of the value of the untreated tooth).

FIG. 3: Microhardness after erosion of the enamel disks from Groups 1 and 2 by a cola lemonade (in percent of the value of the untreated tooth).

FIG. 4 (a+b): Representative electron micrographs of a cross-section of an enamel sample treated with toothpaste according to the invention.

### DETAILED DESCRIPTION OF THE INVENTION

The invention thus relates to the use of a composition comprising at least one poorly water-soluble calcium salt and/or a composite material thereof for the protection and/or for the therapeutic and/or preventive treatment of teeth and/or bones in the case of or against damage which can be attributed to external influences, in particular, of body-related, chemical, physical and/or microbiological type.

The enamel normally contains prism-like structures, whose interprismatic regions show a slightly different dissolution behavior in the case of softening than the prisms themselves. Because of the roughness thus resulting, the enamel becomes susceptible to colonization by microorganisms and/or the attack of acids, in particular, from microbial activity or foods.

By means of the use according to the invention, it is possible to heal the roughness mentioned with tooth-analogous material. What is involved here is not a smoothing of the enamel customary according to the prior art by polishing, but the ordered formation of new, tooth-analogous material (neomineralization). In particular, it is favorable to employ the compositions to be used according to the invention in the case of damaged, softened enamel.

If, due to body-related influences, such as, for example, in the case of pathological dryness of the mouth (xerostomatia) or decreased flow of saliva (hyposalivation), the normal processes in the oral cavity are disordered and can now no longer be used for the restoration and strengthening of the enamel by endogenous forces, the use of a composition used according to the invention is particularly important in order to avoid or to diminish long-term damage to the teeth.

According to a preferred embodiment, the external influences mentioned are, in particular, to be understood as meaning attacks by acids.

The term “acids” is to be understood here both as meaning the intrinsic acids and the extrinsic acids.

The damage due to intrinsic acids relates, as already mentioned, in particular, to medically related syndromes which are accompanied by contact of gastric acid with the oral region, in particular, in the case of eructation, regurgitation, heartburn or due to vomiting, also in connection with pathological eating disorders, such as, in particular, bulimia.

Use in the case of attack by extrinsic acids is particularly suitable, in particular, because of bacterial activity or the action of acids from foods.

Furthermore, it was possible to observe that in addition to the prevention against erosion of teeth (dentine and enamel) and bones, in particular, enamel, repair of erosions on teeth and bones, in particular, the enamel, takes place by means of the use according to the invention.

The use according to the invention, therefore, can also be applied therapeutically in the repair of damage, in particular, of erosion, to bones and teeth, in particular, to the enamel.

After acid damage (etching) of the enamel, a toothpaste used according to the invention led to a very great, almost complete restoration of the enamel surface even after treatment once with the composition according to the invention. The compositions to be used according to the invention are thus particularly suitable for the prevention and repair of erosions.

It has furthermore been found that the poorly water-soluble calcium salts to be used according to the invention and/or their composite materials are able to allow a layer to grow on the teeth, in particular, on the enamel, which seals off the enamel from the oral environment.

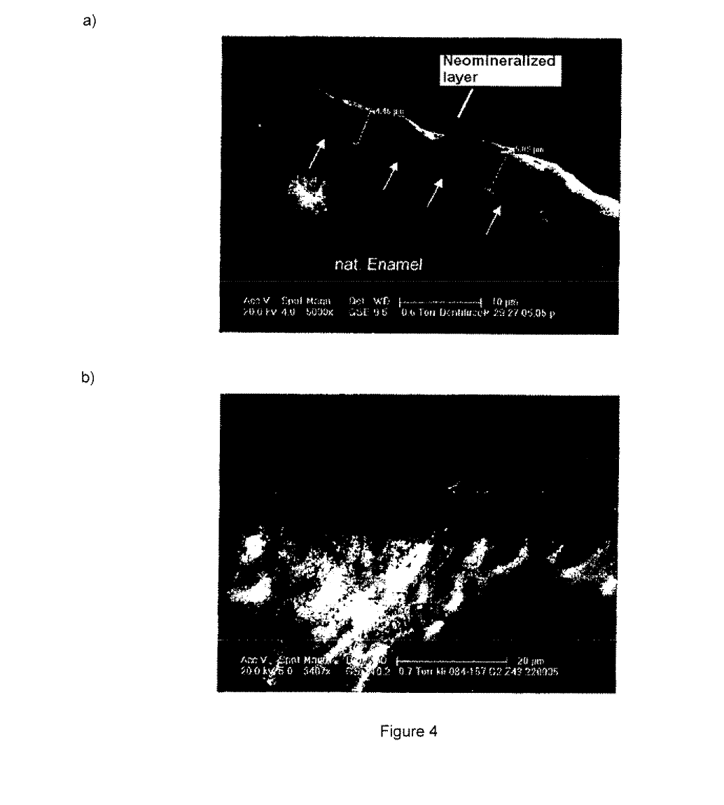

The resulting layer is structured in and intimately bonded to the natural enamel (cf. FIG. 4a+b) and is suitable, in particular, as a “sacrificial layer,” which in the case of acid attack on the teeth is attacked and possibly slightly eroded instead of the enamel. The underlying tooth's own enamel is thereby particularly well protected.

In particular, according to the invention the uses of the teaching according to the invention in toothpastes and other oral cleansing compositions and/or dentifrices are preferred.

According to a preferred use, the protection mentioned and/or the preventive treatment mentioned, leads to a decrease or inhibition of demineralization of the teeth.

According to a further preferred use, the protection mentioned and/or the preventive treatment mentioned leads to a sealing of fissures.

According to the invention, fissures (i.e., the gap-like retractions on the chewing surfaces of the buccal and molar teeth) can be sealed by means of the use according to the invention and thus their susceptibility to caries can be decreased.

According to a preferred use, the protection mentioned and/or the preventive treatment mentioned leads to protection against and/or to the repair of primary lesions and/or initial caries in the enamel.

Primary lesions are understood here in particular, as meaning deep-going demineralization of the dental material, in particular, of the enamel, in the course of initial caries. Because of the establishment of bacteria and their metabolic processes, a carious infection results.

According to a preferred use, the protection mentioned and/or the preventive treatment mentioned leads to the smoothing of the tooth surface.

By means of the use according to the invention, a layer can, in particular, grow on the endogenous tooth material, which has a particularly smooth surface. This leads to a more glossy surface, which is easier to clean and can be colonized with greater difficulty by microorganisms. An improved esthetic impression is produced by the smoothing of the surface, which allows the teeth to appear whiter or lighter.

According to a preferred use, the protection mentioned and/or the preventive treatment mentioned leads to the improvement of the cleanability of the teeth.

According to a preferred use, the protection mentioned and/or the preventive and/or therapeutic treatment mentioned leads to the improvement of the health of the teeth generally.

According to a preferred use, the protection mentioned and/or the preventive treatment mentioned leads to better mechanical resistance, in particular, a decrease of the extent of microscratches, microcraters or mechanical abrasion.

The use according to the invention leads to better resistance to mechanical stress of the teeth, which, in addition to chewing, can also be produced by vigorous toothbrushing. Damage to or abrasion of softened enamel can thus be avoided.

Calcium salts which are poorly soluble in water can be understood as meaning those salts which are soluble to less than 0.1% by weight (1 g/l) in water at 20° C. Suitable salts of this type are, for example, calcium hydroxyphosphate (Ca₅-[OH(PO₄)₃]) or hydroxy-apatite, calcium fluorophosphate (Ca₅[F(PO₄)₃]) or fluoroapatite, fluorine-doped hydroxyapatite of the composition (Ca₅(PO₄)₃(OH,F) and calcium fluoride (CaF₂) or fluorite or fluorspar, and other calcium phosphates such as di-, tri- or tetracalcium phosphate (Ca₂P₂O₇, Ca₃(PO₄)₂, Ca₄P₂O₉, oxyapatite (Ca₁₀(PO₄)₆O) or nonstoichiometric hydroxyapatite (Ca_{5-1/2(x+y)}(PO₄)₃₋ₓ(HPO₄)ₓ(OH)_{1-y}). Likewise suitable are carbonate-containing calcium phosphates (e.g., (Ca_{5-1/2(x+y+z)}(PO₄)_{3-x-z}(HPO₄)ₓ(CO₃)_{z}(OH)_{1-y}), calcium hydrogenphosphate (e.g., CaH(PO₄)ₓ2H₂O) and octacalcium phosphate (e.g., Ca₈H₂(PO₄)₆×5H₂O).

As a calcium salt, one or alternatively two or more salts as a mixture, can be present in the composite materials according to the invention, selected from the group consisting of phosphates, fluorides and fluorophosphates, which can alternatively additionally contain hydroxyl and/or carbonate groups.

According to a preferred embodiment, the poorly water-soluble calcium salt (even in the composite materials) is present in the form of individual crystallites or in the form of particles, comprising a plurality of said crystallites, the mean particle diameter being below 1,000 nm, preferably in the range between 10 and 300 nm.

The particle diameter should be understood here as meaning the diameter of the particles (crystallites or particles) in the direction of their greatest longitudinal extension. The mean particle diameter is to be understood as meaning a value averaged over the total amount of the composite. According to the invention, it is below 1,000 nm, preferably below 300 nm.

Preferably, the mean particle diameter of the crystallites is in the range from 10 to 150 nm, and particularly preferably the crystallites are present with a thickness in the range from 2 to 50 nm and a length in the range from 10 to 150 nm. Thickness is to be understood here as meaning the smallest diameter of the crystallites and length, their largest diameter.

The particle diameter of the crystallites can be determined by methods familiar to the person skilled in the art, in particular, by the evaluation of the scattering of the reflections observed in X-ray diffraction. Preferably, evaluation is carried out here by fit processes, for example, the Rietveld method.

Preferably, a single crystallite has a thickness in the range from 2 to 50 nm and a length in the range from 10 to 150 nm, preferably a thickness of 2 to 15 nm and a length of 10 to 50 nm, particularly preferably a thickness of 3 to 11 nm and a length of 15 to 25 nm. Thickness is to be understood here as meaning the smallest diameter of the crystallites and length, their largest diameter.

According to a particularly preferred embodiment, the particles of the calcium salts are rod-shaped and/or lamellar.

According to a particularly preferred embodiment, the particles of the calcium salts according to the invention (even in the composite materials) have an elongated, in particular, rod-like or needle-like, shape. This has the particular advantage that they are very similar to the shape of the biological apatites (e.g., bone and dentine apatites) and, therefore, have a particularly good capability for re- and neomineralization.

According to another particularly preferred embodiment, the particles of the calcium salts are preferably mainly lamellar.

Surprisingly, it was possible to produce composite materials which have a mainly lamellar structure of the poorly water-soluble calcium salt and thus can imitate the bone substance particularly well.

In the context of the present invention, mainly lamellar means that at least 50%, preferably at least 70%, particularly preferably at least 80%, of the particles of the calcium salts are present in the form of platelets. Particularly preferably, the particles have an essentially lamellar shape.

Advantageously, the composite materials according to the invention containing the mainly lamellar calcium particles are particularly similar to the structure of the bone substance in vivo, which is likewise constructed from plates. This has the particular advantage that because of the similarity of the shape to the biological apatites (e.g., bone or dentine apatite), the composite materials according to the invention have a particularly good capability for re- and neomineralization, such that the process of biomineralization can take place even more rapidly and effectively.

A further advantage of the invention consists in the fact that the composite materials with a mainly lamellar structure of the calcium salts have improved biocompatibility.

According to a particular embodiment, the lamellar particles have a breadth in the range from 5 to 150 nm and a length in the range from 10 to 150 nm and a height (thickness) of 2 to 50 nm.

According to a very particularly preferred embodiment, the average length of the particles is preferably 30 to 100 nm. Preferably, the breadth of these particles here is in the range between 10 and 100 nm.

The particle diameter of the particles can be determined by methods familiar to the person skilled in the art, in particular, by the utilization of imaging processes, in particular, transmission electron microscopy.

Height (thickness) is to be understood here as meaning the smallest diameter of the particles based on the three directions in space perpendicular to one another, length being their greatest diameter. The breadth of the particles is accordingly the further diameter perpendicular to the length which is equal to or less than the longitudinal dimension of the particle, but greater or at least equal to its height dimension.

The lamellar particles are present as more or less irregularly shaped particles, in some cases as rather round, in some cases rather angular particles, and also with rounded edges. This may be observed most clearly in images recorded under transmission electron microscopy.

The lamellar particles are often also present repeatedly overlapping in samples of this type. Overlapping particles are generally pictured with a greater darkening at the sites of overlapping than non-overlapping particles. The lengths, breadths and heights indicated are preferably determined (measured) on non-overlapping particles of the sample.

The height of the lamellar particles can preferably be obtained from recordings of this type by the determination of the dimensions of the particles with their largest surface perpendicular to the image plane. The particles perpendicular to the image plane are distinguished by a particularly high contrast (high darkening) and appear rather rod-like here. These lamellar particles perpendicular to the image plane can be identified as actually perpendicular to the image plane if they show a broadening of the dimension (at least in one direction in space) on tilting of the image plane and a decrease in the darkening of the picture.

For the determination of the height of the particles, it is particularly suitable to tilt the image plane of the sample repeatedly in various positions and to determine the dimensions of the particles in the adjustment, which is characterized by the highest contrast/highest darkening and the smallest dimension of the particles. The shortest dimension then corresponds here to the height of the particles.

According to a particular embodiment, in the case of the particles of the composite materials according to the invention the ratio of length to breadth is between 1 and 4, preferably from 1 to 3, particularly preferably between 1 and 2, for example, 1.2 (length 60 nm, breadth 50 nm) or 1.5 (length 80, breadth 40 nm).

The lamellar shape of the particles is formed by the ratio of length to breadth. If the ratio between length and breadth is markedly greater than 4, rod-like particles are more likely present.

The advantage of the lamellar particles with a ratio of preferably 1 to 2 lies in the fact that these particles have a particularly similar length to breadth ratio to the natural bone material and, therefore, have a particularly good and biologically compatible re- or neomineralization of the tooth material (dentine and enamel).

According to a further particular embodiment, the particles have an area of 0.1×10⁻¹⁵ m² to 90×10⁻¹⁵ m², preferably an area of 0.5×10⁻¹⁵ m² to 50×10⁻¹⁵ m², particularly preferably 1.0×10⁻¹⁵ m² to 30×10⁻¹⁵ m², very particularly preferably 1.5×10⁻¹⁵ m² to 15×10⁻¹⁵ m², for example, 2×10⁻¹⁵ m².

The area of the particles determined is the area of the plane opened up by the length and the breadth perpendicular to it, determined by the customary geometrical calculation methods.

Surprisingly, it is possible with the present invention to produce the composite materials according to the invention in the form of crystalline inorganic nanoparticles which lead to a particularly effective neomineralization of dental material (dentine and enamel) and bone tissue.

According to a preferred embodiment, composite materials according to the invention are employed which are selected from
a) at least one calcium salt which is poorly soluble in water; and
b) a polymer component.

Composite materials are understood as meaning compounds which comprise the components mentioned under a) and b) and represent aggregates which appear microscopically heterogeneous, but macroscopically homogeneous, and in which the calcium salts, preferably in the form of individual crystallites or in the form of particles comprising a majority of said crystallites, are present in associated form in the structure of the polymer component. The proportion of the polymer components in the composite materials is between 0.1 and 80% by weight, but preferably between 10 and 60% by weight, in particular, between 30 and 50% by weight, based on the total weight of the composite materials.

According to a particular embodiment, the polymer component is selected from a protein component, polyelectrolytes and polysaccharides.

A preferred embodiment of the invention consists in employing polyelectrolytes as the polymer component. Suitable polyelectrolytes within the meaning of the invention are polyacids and polybases, where the polyelectrolytes can be biopolymers or alternatively synthetic polymers. Thus, the compositions according to the invention contain, for example, one or alternatively more polyelectrolytes selected from
alginic acids
pectins
carrageenans
polygalacturonic acids
amino and amino acid derivatives of alginic acids, pectins, carrageenans and polygalacturonic acids
polyamino acids, such as, for example, polyaspartic acids
polyaspartamides
nucleic acids, such as, for example, DNA and RNA
lignosulfonates
carboxymethylcelluloses
amino- and/or carboxyl group-containing cyclodextrin, cellulose or dextran derivatives
polyacrylic acids
polymethacrylic acids
polymaleates
polyvinylsulfonic acids
polyvinylphosphonic acids
polyethyleneimines
polyvinylamines
   and derivatives of the above-mentioned substances, in particular, amino and/or carboxyl derivatives. Preferably, in the context of the present invention polyelectrolytes are employed which carry groups suitable for salt formation with divalent cations. Carboxylate group-bearing polymers are particularly suitable.

Within the meaning of the invention, particularly preferred polyelectrolytes are poly-aspartic acids, alginic acids, pectins, deoxy-ribonucleic acids, ribonucleic acids, polyacrylic acids and polymethacrylic acids.

Polyaspartic acids having a molecular weight in the range between about 500 and 10,000 daltons, in particular, 1,000 to 5,000 daltons, are very particularly preferred.

Another preferred embodiment of the invention consists in selecting polysaccharides as the polymer component. In particular, these polysaccharides are selected from glucuronic acid and/or iduronic acid-containing polysaccharides. Among these are to be understood those polysaccharides which, inter alia, are synthesized from glucuronic acid, preferably D-glucuronic acid and/or iduronic acid, in particular, L-iduronic acid. One constituent of the carbohydrate skeleton is formed here from glucuronic acid or iduronic acid. The iduronic acid isomeric to glucuronic acid has the other configuration on the C5 carbon atom of the ring. Preferably, glucuronic acid- and/or iduronic acid-containing polysaccharides are to be understood as meaning those polysaccharides which contain glucuronic acid and/or iduronic acid in a molar ratio of 1:10 to 10:1, in particular, of 1:5 to 5:1, particularly preferably 1:3 to 2:1, based on the sum of the further monosaccharide structural units of the polysaccharide. Advantageously, a particularly good interaction with the calcium salt can be achieved by the anionic carboxyl groups of the glucuronic acid- and/or iduronic acid-containing polysaccharides, which leads to a particularly stable and simultaneously particularly well biomineralizing composite material. For example, suitable polysaccharides are the glucuronic acid- and/or iduronic acid-containing glycosaminoglycans (also designated as mucopoly-saccharides), microbially produced xanthan or welan or gum arabic, which is obtained from acacias.

One advantage of the composite materials according to the invention is the particular stability in aqueous systems even without the addition of dispersing aids such as, for example, polyhydric alcohols (such as glycerol or polyethylene glycols).

According to a particularly preferred embodiment, the polymer component is selected from a protein component, preferably from proteins, protein hydrolyzates and their derivatives.

In principle, possible proteins in the context of the present invention are all proteins independently of their origin or their preparation. Examples of proteins of animal origin are keratin, elastin, collagen, fibroin, albumin, casein, whey protein, placental protein. Of these, collagen, keratin, casein and whey protein are preferred according to the invention. Proteins of plant origin such as, for example, wheat and wheatgerm protein, rice protein, soybean protein, oat protein, pea protein, potato protein, almond protein and yeast protein can likewise be preferred according to the invention.

Protein hydrolyzates are to be understood in the context of the present invention as meaning degradation products of proteins such as, for example, collagen, elastin, casein, keratin, almond, potato, wheat, rice and soybean protein, which are obtained by acidic, alkaline and/or enzymatic hydrolysis of the proteins themselves or their degradation products such as, for example, gelatin. For enzymatic degradation, all enzymes acting hydrolytically are suitable, such as, for example, alkaline proteases. Further suitable enzymes and enzymatic hydrolysis processes are described, for example, in K. Drauz and H. Waldmann, Enzyme Catalysis in Organic Synthesis, VCH Verlag, Weinheim 1975. During the degradation, the proteins are cleaved into smaller subunits, where the degradation can proceed through the stages of the polypeptides through the oligopeptides down to the individual amino acids. The only slightly degraded protein hydrolyzates include, for example, the gelatins preferred in the context of the present invention, which can have molar masses in the range from 15,000 to 250,000 D. gelatin, is a polypeptide which is principally obtained by hydrolysis of collagen under acidic (gelatin type A) or alkaline (gelatin type B) conditions. The gel strength of the gelatin is proportional to its molecular weight, i.e., a more strongly hydrolyzed gelatin affords a lower viscosity solution. The gel strength of the gelatin is indicated in Bloom numbers. During the enzymatic cleavage of the gelatin, the polymer size is greatly reduced, which leads to very low Bloom numbers.

Furthermore, preferred protein hydrolyzates in the context of the present invention are the protein hydrolyzates customary in cosmetics having an average molecular weight in the range from 600 to 4,000, particularly preferably from 2,000 to 3,500. Summaries of preparation and use of protein hydrolyzates have been published, for example, by G. Schuster and A. Domsch in Seifen Öle Fette Wachse 108, (1982) 177 or Cosm. Toil. 99, (1984) 63, by H. W. Steisslinger in Parf. Kosm. 72, (1991) 556 and F. Aurich et al. in Tens. Surf. Det. 29, (1992) 389. Preferably, protein hydrolyzates according to the invention of collagen, keratin, casein and plant proteins are employed, for example, those based on wheat gluten or rice protein, whose preparation is described in the two German patent specifications DE 19502167 C1 and DE 19502168 C1 (Henkel).

Protein hydrolyzate derivatives are understood in the context of the present invention as meaning chemically and/or chemoenzymatically modified protein hydrolyzates such as, for example, the compounds known under the INCI names sodium cocoyl hydrolyzed wheat protein, laurdimonium hydroxypropyl hydrolyzed wheat protein, potassium cocoyl hydrolyzed collagen, potassium undecylenoyl hydrolyzed collagen and laurdimonium hydroxypropyl hydrolyzed collagen. Preferably, derivatives according to the invention of protein hydrolyzates of collagen, keratin and casein and also plant protein hydrolyzates are employed, such as, for example, sodium cocoyl hydrolyzed wheat protein or laurdimonium hydroxypropyl hydrolyzed wheat protein.

Further examples of protein hydrolyzates and protein hydrolyzate derivatives which come under the context of the present invention are described in CTFA 1997 International Buyers' Guide, John A. Wenninger et al. (Ed.), The Cosmetic, Toiletry, and Fragrance Association, Washington, D.C. 1997, 686-688.

In each of the composite materials according to the invention, the protein component can be formed by one or more substances selected from the group consisting of proteins, protein hydrolyzates and protein hydrolyzate derivatives.

Preferred protein components are to be understood as meaning all structure-forming proteins, protein hydrolyzates and protein hydrolyzate derivatives, which because of their chemical constitution form certain three-dimensional spatial structures which are familiar to the person skilled in the art from protein chemistry under the terms of secondary, tertiary or alternatively, quaternary structure.

According to a particularly preferred use, the protein component of the composite materials is selected from gelatin and casein and their hydrolyzates.

According to a particularly preferred embodiment, gelatins of the type AB can be employed, which are also known under the name “acid-bone” or “acid process ossein” gelatins, and are prepared from ossein by means of strongly acidic process conditions.

Ossein, as a collagen-containing starting material for the preparation of gelatins of the type AB “acid-bone” or “acid process ossein,” is prepared as an extract of comminuted bones, in particular, cattle bones, which are optionally stored in aqueous solution, preferably cold acid, preferably dilute acid (e.g., hydrochloric acid) for one or more days (preferably at least one week or more) after defatting and drying in order to remove the inorganic bone constituents, in particular, hydroxyapatite and calcium carbonate. A spongy demineralized bone material, ossein, results.

The collagen found in the ossein is denatured by a hydrolysis process and released by treating the material under strongly acidic conditions.

The preparation of the gelatin from the raw materials mentioned takes place by repeated extraction with aqueous solutions. Preferably, the pH of the solution can be adjusted before the extraction process. A number of extraction steps with water or aqueous solutions with increasing solvent temperature are particularly preferred.

Composite materials which can be obtained from a poorly water-soluble calcium salt with the gelatins of type AB (acid-bone) are particularly suitable for employment in uses according to the invention.

The composite materials according to the invention are thus structured composite materials in contrast to the composite of hydroxyapatite and collagen described in R. Z. Wang et al., in which uniformly dispersed hydroxyapatite nanoparticles are present. A further significant difference between the subject of the present invention and the prior art consists in the size and morphology of the inorganic component. The hydroxyapatite particles present in the hydroxyapatite/collagen composite described in R. Z. Wang et al. have a size of 2-10 nm. Hydroxyapatite particles in this size range are to be counted among the range of the amorphous or partially X-ray-amorphous substances.

In a further embodiment of the invention, in the composition the mentioned crystallites or particles of the calcium salts present can be covered by one or more surface modification agents.

By means of this, for example, the production of composite materials can be facilitated in those cases in which the calcium salts can be poorly dispersed. The surface modification agent is adsorbed on the surface of the calcium salts and modifies it in such a way that the dispersibility of the calcium salt increases and the agglomeration of the nanoparticles is prevented.

The structure of the composite materials and the loading of the polymer component with the nanoparticulate calcium salt can be influenced by surface modification. In this way, it is possible during the use of the composite materials in remineralization processes to exert influence on the course and the rate of the remineralization process.

Surface modification agents are to be understood as meaning substances which adhere to the surface of the finely divided particles, but do not chemically react with these. The molecules of the surface modification agent adsorbed individually on the surface are essentially free of intermolecular bonds with one another. Surface modification agents are, in particular, understood as meaning dispersants. Dispersants are also known to the person skilled in the art, for example, under the terms emulsifiers, protective colloids, wetting agents, detergents etc.

Possible surface modification agents are, for example, emulsifiers of the type consisting of the nonionic surfactants from at least one of the following groups:
addition products of 2 to 30 mol of ethylene oxide and/or 0 to 5 mol of propylene oxide to linear fatty alcohols having 8 to 22 C atoms, to fatty acids having 12 to 22 C atoms and to alkylphenols having 8 to 15 C atoms in the alkyl groups;
C_{12/18}-fatty acid mono- and diesters of addition products of 1 to 30 mol of ethylene oxide to glycerol;
glycerol mono- and diesters and sorbitan mono- and diesters of saturated and unsaturated fatty acids having 6 to 22 carbon atoms and their ethylene oxide addition products;
alkyl mono- and oligoglycosides having 8 to 22 carbon atoms in the alkyl radical and their ethoxylated analogs;
addition products of 15 to 60 mol of ethylene oxide to castor oil and/or hardened castor oil;
polyol and in particular, polyglycerol esters, such as, for example, polyglycerol polyricinoleate, polyglycerol poly-12-hydroxystearate or poly-glycerol dimerate. Mixtures of compounds of a number of these substance classes are likewise suitable;
addition products of 2 to 15 mol of ethylene oxide to castor oil and/or hardened castor oil;
partial esters based on linear, branched, unsaturated or saturated C_{6/22}-fatty acids, ricinoleic acid and 12-hydroxystearic acid and glycerol, polyglycerol, pentaerythritol, dipenta-erythritol, sugar alcohols (e.g., sorbitol), alkyl glucosides (e.g., methyl glucoside, butyl glucoside, lauryl glucoside) and polyglucosides (e.g., cellulose);
mono-, di- and trialkyl phosphates and mono-, di- and/or tri-PEG-alkyl phosphates and their salts; wool wax alcohols;
polysiloxane/polyalkyl polyether copolymers or corresponding derivatives;
mixed esters of pentaerythritol, fatty acids, citric acid and fatty alcohol according to DE-C 1165574 and/or mixed esters of fatty acids having 6 to 22 carbon atoms, methylglucose and polyols, preferably glycerol or polyglycerol; and
polyalkylene glycols.

The addition products of ethylene oxide and/or of propylene oxide to fatty alcohols, fatty acids, alkylphenols, glycerol mono- and diesters, and sorbitan mono- and diesters of fatty acids or to castor oil are known, commercially obtainable products. What is involved here are mixtures of homologs, whose mean degree of alkoxylation corresponds to the ratio of amounts of ethylene oxide and/or propylene oxide and substrate material with which the addition reaction is carried out.

C_{8/18}-alkyl mono- and oligoglycosides, their preparation and their use are known from the prior art. Their preparation is carried out, in particular, by reaction of glucose or oligosaccharides with primary alcohols having 8 to 18 C atoms. With respect to the glycoside radical, both monoglycosides in which a cyclic sugar radical is bonded glycosidically to the fatty alcohol, and oligomeric glycosides having a degree of oligomerization up to preferably approximately 8, are suitable. The degree of oligomerization here is a statistical mean value, on which a customary homolog distribution for technical products of this type is based.

Typical examples of anionic emulsifiers are soaps, alkylbenzenesulfonates, alkanesulfonates, olefinsulfonates, alkyl ether sulfonates, glycerol ether sulfonates, α-methyl ester sulfonates, sulfo-fatty acids, alkylsulfates, alkyl ether sulfates such as, for example, fatty alcohol ether sulfates, glycerol ether sulfates, hydroxy mixed ether sulfates, monoglyceride (ether) sulfates, fatty acid amide (ether) sulfates, mono- and dialkyl sulfo-succinates, mono- and dialkyl sulfosuccinamates, sulfo-triglycerides, amide soaps, ethercarboxylic acids and their salts, fatty acid isethionates, fatty acid sarcosinates, fatty acid taurides, N-acylamino acids such as, for example, acyl glutamates and acyl aspartates, alkyl oligoglucoside sulfates, protein fatty acid condensates (in particular, plant products based on wheat), and alkyl (ether) phosphates. If the anionic surfactants contain polyglycol ether chains, these can have a conventional, but preferably a concentrated, homolog distribution.

Zwitterionic surfactants can also be used as emulsifiers. Zwitterionic surfactants are designated as those surface-active compounds which carry at least one quaternary ammonium group and at least one carboxylate and one sulfonate group in the molecule. Particularly suitable zwitterionic surfactants are the “betaines” such as the N-alkyl-N,N-dimethylammonium glycinates, for example, coconut alkyl dimethylammonium glycinate, N-acylaminopropyl-N,N-dimethylammonium glycinates, for example, coconut acylaminopropyldimethylammonium glycinate, and 2-alkyl-3-carboxyl-methyl-3-hydroxyethylimidazolines in each case having 8 to 18 C atoms in the alkyl or acyl group, and coconut acylaminoethylhydroxyethylcarboxymethyl glycinate. The fatty acid amide derivative known under the CTFA name cocamidopropylbetaine is particularly preferred. Emulsifiers which are likewise suitable are ampholytic surfactants. Ampholytic surfactants are understood as meaning those surface-active compounds which, aside from a C_{8/18}-alkyl or -acyl group in the molecule, contain at least one free amino group and at least one —COOH group or —SO₃H group and are capable of the formation of internal salts. Examples of suitable ampholytic surfactants are N-alkylglycines, N-alkyl-propionic acids, N-alkylaminobutyric acids, N-alkyl-iminodipropionic acids, N-hydroxyethyl-N-alkylamido-propylglycines, N-alkyltaurines, N-alkylsarcosines, 2-alkylaminopropionic acids and alkylaminoacetic acids in each case having approximately 8 to 18 C atoms in the alkyl group. Particularly preferred ampholytic surfactants are N-coconut alkylaminopropionate, coconut acylaminoethylaminopropionate and C_{12/18}-acylsarcosine. In addition to the ampholytic emulsifiers, quaternary emulsifiers are also suitable, where those of the type consisting of the ester quats, preferably methyl-quaternized difatty acid triethanolamine ester salts, are particularly preferred.

Protective colloids suitable as surface modification agents are, for example, natural water-soluble polymers such as, for example, gum arabic, starch, water-soluble derivatives of water-insoluble polymeric natural substances such as, for example, cellulose ethers such as methylcellulose, hydroxyethyl-cellulose, carboxymethylcellulose or modified carboxy-methylcellulose, hydroxyethylstarch or hydroxypropyl-guar, and synthetic water-soluble polymers, such as, for example, polyvinyl alcohol, polyvinylpyrrolidone, polyalkylene glycols, polyaspartic acid and poly-acrylates.

Generally, the surface modification agents are employed in a concentration of 0.1 to 50, but preferably 1 to 20% by weight, based on the calcium salts.

Preferably suitable surface modification agents are especially the nonionic surfactants in an amount from 1 to 20% by weight, based on the weight of the calcium salt. The nonionic surfactants of the type consisting of the alkyl C₈-C₁₆-(oligo)glucosides and the ethoxylates of hardened castor oil have proven particularly effective.

The composite materials according to the invention can be prepared by precipitation reactions of aqueous solutions of water-soluble calcium salts and aqueous solutions of water-soluble phosphates and/or fluoride salts, the precipitation being carried out in the presence of polymer components. This is preferably carried out in such a way that the polymer components are added before the precipitation reaction in pure, dissolved or colloidal form of the neutral or alkaline aqueous phosphate and/or fluoride salt solution or of the neutral or alkaline or acidic solution of the calcium salt. Alternatively, the polymer components can be introduced in pure, dissolved or colloidal form and subsequently treated with the neutral or alkaline or acidic calcium salt solution and the neutral or alkaline phosphate and/or fluoride salt solution in succession in any desired sequence or simultaneously. Neutral solutions should be understood as meaning solutions having a pH of between approximately 6.5 and approximately 7.5.

In the preparation process according to the invention, the combination of the individual components can, in principle, take place in all possible sequences. The alkalizing agent used is preferably ammonia.

A further variant according to the invention of the preparation process consists in the fact that the precipitation of an acidic solution of a water-soluble calcium salt together with a stoichiometric amount of a water-soluble phosphate and/or fluoride salt or of an acidic solution of hydroxyapatite having a pH of below 5, preferably at a pH below 3, is carried out by raising the pH with aqueous alkali or ammonia in the presence of the polymer components.

A further process variant consists in the fact that nanoparticulate calcium salts in pure or dispersed form or dispersions of nanoparticulate calcium salts prepared from aqueous solutions of water-soluble calcium salts and aqueous solutions of water-soluble phosphate and/or fluoride salts by precipitation reactions are treated with the polymer components, the latter preferably in dissolved or dispersed form, where any desired sequence can be chosen during the addition.

Preferably, the solution or dispersion of the polymer component is introduced and a dispersion of the nanoparticulate calcium salt is added.

In all preparation processes mentioned, the resulting dispersion of the composite material can be separated off from the solvent and the other constituents of the reaction mixture if required by processes known to the person skilled in the art, such as, for example, filtration or centrifugation and isolated in solvent-free form by subsequent drying, e.g., by freeze-drying.

The solvent used in all preparation processes is preferably water, but in individual steps of the preparation, organic solvents such as, for example, mono- or polyhydric alcohols having 1 to 4 C atoms or glycerol can also be used.

The preparation of the composite materials according to the invention, in which the primary particles of the calcium salts are surface-modified, can be carried out according to analogous precipitation processes as described above, where, however, the precipitation of the nanoparticulate calcium salts or of the composite materials is carried out in the presence of one or more surface modification agents.

Preferably, the surface-modified nano-particulate calcium salts are first produced by a precipitation reaction between aqueous solutions of calcium salts and aqueous solutions of phosphate and/or fluoride salts in the presence of surface modification agents. These calcium salts can subsequently be purified from accompanying products of the reaction mixture, e.g., by concentrating under reduced pressure and subsequent dialysis. By stripping off the solvent, a dispersion of the surface-modified calcium salt can additionally be prepared with a solids content as desired. Subsequently, the composite material consisting of surface-coated calcium salt and polymer components is formed by addition of the polymer components in pure, dissolved or colloidal form, the sequence of the addition in turn being uncritical, and if necessary after reaction at elevated temperature, preferably in the range between 50 and 100° C. and for a period of 1 to 100 minutes.

Further processes can be used for the production of dispersions of surface-modified calcium salts, as described in German application DE 19858662.0.

Such calcium salt particles, which have an elongated, in particular, rod- or needle-like form, can, in particular, be prepared by processes according to the invention in which the precipitation of the composite material is carried out at a pH between approximately 9.5 and 14, preferably between 10 and 12, preferably about pH 11.

A process in which the precipitation of the composite material is carried out at a pH between 5 and 9, preferably between 6 and 8, particularly preferably about 7, is particularly suitable for the production of a composite material according to the invention, which mainly contains lamellar particles.

Preferably, for the formation of the preferred composite material according to the invention a solution of a calcium salt is introduced here with the polymer component and a phosphate solution is slowly added, the pH being between 5 and 9, preferably between 6 and 8, particularly preferably about 7. Particularly preferably, the pH during the addition of the phosphate solution is kept constant by addition of corresponding amounts of aqueous base.

The present use according to the invention of poorly water-soluble calcium salts and/or their composite materials is, therefore, suitable for use in compositions for the prevention of erosion on bones and teeth, in particular, enamel, for the protection of the teeth from attack by acids, in particular, by bacterial activity or by the action of acids from foods, for the repair of erosion symptoms on teeth, for protection against demineralization of the teeth, for the sealing of fissures, for the repair of primary lesions and/or initial caries in the enamel and for the smoothing of the tooth surface, for caries prophylaxis, for the improvement of the cleanability and mechanical resistance of the teeth, and dental health generally.

Preferably, at least 0.000001% by weight, particularly preferably 0.0001 to 80% by weight, in particular, 0.001 to 10% by weight, especially preferably 0.01 to 5% by weight, very particularly preferably 0.01 to 4% by weight, of at least one poorly water-soluble calcium salt and/or its composite material of such a preparation are present.

These preparations are, in particular, selected from oral and tooth care compositions and oral cleaning compositions and dentifrices and candies.

The compositions for the cleaning and care of the teeth can be present here, for example, in the form of pastes, liquid creams, gels or mouthwashes. Even in liquid preparations, the composite materials suitable for the use according to the invention disperse easily, remain stably dispersed and are not prone to sedimentation.

The content of the poorly water-soluble calcium salt or its composite materials in the mouth and tooth care compositions used according to the invention is 0.01 to 10% by weight, preferably 0.01 to 2% by weight, based on the total weight of the composition.

Furthermore, the oral and tooth care compositions according to the invention can contain 0.1 to 9% by weight, in particular, 2 to 8% by weight, of at least one polishing agent.

Polishing agents belong to the essential constituents of a tooth gel and are present, depending on their intended function, alone or in combination with other polishing compounds or polishing agents. They are used for the mechanical removal of the uncalcified plaque and should ideally lead to the imparting of gloss to the tooth surface (polishing effect) together with simultaneous minimal scouring action (abrasion effect) and damage to the enamel and the dentine. The abrasion behavior of the polishing agents and polishing substances is essentially determined by their hardness, particle size distribution and surface structure. In the selection of suitable polishing compounds, consequently those substances are in particular, preferably selected which have minimal abrasive action together with high cleaning power.

Nowadays, substances which have small particle sizes, are largely free of sharp corners and edges and whose hardness and mechanical properties do not stress the tooth or the tooth substance excessively are mainly used as cleaning compounds.

Customarily, water-insoluble inorganic substances are employed as cleaning compounds or polishing agents. The use of very finely divided polishing agents having a mean particle size of 1-200 μm, preferably 1-50 μm and in particular, 1-10 μm is particularly advantageous.

In principle, the polishing agents according to the invention can be selected from silicic acids, aluminum hydroxide, alumina, silicates, organic polymers or mixtures thereof. Furthermore, however, “metaphosphates,” alkaline earth metal carbonates or hydrogencarbonates and calcium-containing polishing components can also be present in the compositions according to the invention.

It can be preferable according to the invention to employ silicic acids as polishing agents in toothpastes or liquid dentifrices. Among the silicic acid polishing agents, a basic distinction is made between gel silicic acids, hydrogel silicic acids and precipitation silicic acids. Precipitation and gel silicic acids are particularly preferred according to the invention, since they can be widely varied in their preparation and are particularly highly compatible with fluoride active substances. Furthermore, they are also particularly well suited for the production of gel or liquid tooth gels.

Gel silicic acids are produced by the reaction of sodium silicate solutions with strong, aqueous mineral acids with formation of a hydrosol, ageing to the hydrogel, washing and subsequent drying. If the drying is carried out under gentle conditions to water contents of 15 to 35% by weight, “hydrogel silicic acids” are obtained, as described, for example, in U.S. Pat. No. 4,153,680. By drying these hydrogel silicic acids to water contents of below 15% by weight, an irreversible shrinkage of the previously loose structure to the tight structure of the “xerogel” takes place. Xerogel silicic acids of this type are known, for example, from U.S. Pat. No. 3,538,230.

A second, preferably suitable group of silicic acid polishing agents are the precipitation silicic acids. These are obtained by precipitation of silicic acid from dilute alkali metal silicate solutions by the addition of strong acids under conditions in which aggregation to give the sol and gel cannot occur. Suitable processes for the preparation of precipitation silicic acids are described, for example, in DE-A 25 22 586 and in DE-A 31 14 493. A precipitation silicic acid prepared according to DE-A 31 14 493 with a BET surface area of 15-110 m²/g, a particle size of 0.5 to 20 μm, where at least 80% by weight of the primary particles should be below 5 μm, and a viscosity in 30% strength glycerol/water (1:1) dispersion of 30-60 Pas (20° C.) in an amount of 10-20% by weight of the toothpaste is particularly suitable according to the invention. Preferably suitable precipitation silicic acids of this type have rounded corners and edges and are obtainable, for example, under the trade name Sident®12 DS from Degussa.

Further precipitation silicic acids of this type are Sident®8 from Degussa and Sorbosil®AC 39 from Crosfield Chemicals. These silicic acids are distinguished by a lower thickening action and a somewhat higher mean particle size of 8-14 μm with a specific surface area of 40-75 m²/g (according to BET) and are particularly suitable for liquid tooth gels. These should have a viscosity (25° C., shear rate D=10 s⁻¹) of 10-100 Pas.

In addition, the silicic acids of the type Zeodent® from Huber-Corp., Tixosil® from Rhodia, and further Sorbosil types can be employed in the compositions according to the invention. Zeodent® 113, Tixosil® 123 and Sorbosil° AC39 are particularly preferred.

Toothpastes which have a markedly higher viscosity of more than 100 Pas (25° C., D=10 s⁻¹), however, need an adequately high proportion of silicic acids having a particle size of less than 5 μm, preferably at least 3% by weight of a silicic acid having a particle size of 1 to 3 μm. In addition to the precipitation silicic acids mentioned, even more finely divided, “thickening silicic acids” having a BET surface area of 150-250 m²/g are, therefore, preferably added to toothpastes of this type. As examples of commercial products which fulfill the conditions mentioned, Sipernat® 22 LS or Sipernat® 320 DS from Degussa may be mentioned in particular.

A suitable alumina polishing agent is preferably a weakly calcined argillaceous earth having a content of α- and γ-alumina in an amount of approximately 0.01 to 5% by weight, preferably 0.1 to 2% by weight, based on the total weight of the composition.

Suitable weakly calcined argillaceous earths are produced from aluminum hydroxide by calcination. Aluminum hydroxide changes by calcination into the α-Al₂O₃ thermodynamically stable at temperatures above 1,200° C. The thermodynamically unstable Al₂O₃ modifications occurring at temperatures between 400 and 1,000° C. are designated as gamma-forms (cf. Ullmann, Encyclopedia of Industrial Chemistry, 4th edition (1974), volume 7, page 298). By selection of the temperature and the length of time in the calcination, the degree of calcination, i.e., the conversion into the thermodynamically stable α-Al₂O₃, can be adjusted to any desired level. By means of weak calcination, an argillaceous earth having a content of γ-Al₂O₃ is obtained which is all the lower, the higher the calcination temperature and the longer the calcination period. Weakly calcined argillaceous earths differ from pure α-Al₂O₃ by a lower hardness of the agglomerates, a greater specific surface area and greater pore volumes.

The dentine abrasion (RDA) of the weakly calcined argillaceous earths to be used according to the invention having a proportion of 10-50% by weight of γ-Al₂O₃ is only 30-60% of the dentine abrasion of a strongly calcined, pure α-Al₂O₃ (measured in a standard toothpaste with 20% by weight of argillaceous earth as the only polishing agent).

In contrast to α-Al₂O₃, γ-Al₂O₃ can be stained red with an aqueous-ammoniac solution of alizarin S (1,2-dihydroxy-9,10-anthraquinone-4-sulfonic acid). The degree of stainability can be chosen as a measure of the degree of calcination or of the proportion of γ-Al₂O₃ in a calcined argillaceous earth:

About 1 g of Al₂O₃, 10 ml of a solution of 2 g/l of alizarin S in water and 3 drops of an aqueous, 10% strength by weight solution of NH₃ are added to a test tube and briefly boiled. The Al₂O₃ is subsequently filtered, washed, dried and assessed under the microscope or analyzed calorimetrically.

Suitable, weakly calcined argillaceous earths having a content of 10-50% by weight of γ-Al₂O₃ can be stained slightly to deeply pink by this process.

Alumina polishing agents of various degrees of calcination, milling fineness and bulk weight are commercially obtainable, e.g., the “polishing argillaceous earths” from Giulini-Chemie or ALCOA.

A preferably suitable quality “Polishing argillaceous earth P10 very fine” has a conglomerate size of under 20 μm, a mean primary crystal size of 0.5-1.5 μm and a bulk weight of 500-600 g/l.

The use of silicates as polishing agent components can likewise be preferred according to the invention. They are particularly employed in modern practice as polishing materials. Examples of silicates employable according to the invention are aluminum silicates and zirconium silicates. The sodium aluminum silicate of the empirical formula Na₁₂(AlO₂)₁₂(SiO₂)₁₂×7H₂O can, in particular, be suitable as a polishing agent, as, for example, the synthetic zeolite A.

Examples of water-insoluble metaphosphates according to the invention are especially sodium metaphosphate, calcium phosphate such as, for example, tricalcium phosphate, calcium hydrogenphosphate, calcium hydrogenphosphate dihydrate and calcium pyrophosphate.

In addition, according to the invention magnesium carbonate, magnesium hydrogenphosphate, trimagnesium phosphate or sodium hydrogencarbonate can be employed as polishing agents, in particular, as a mixture with other polishing agents.

A further polishing agent which is suitable for use in the mouth and tooth care compositions according to the invention is calcium phosphate dihydrate (CaHPO₄×2H₂O). Calcium phosphate dihydrate occurs in nature as brushite and is commercially obtainable as a polishing agent in suitable particle sizes of 1 to 50 μm.

Oral and tooth care compositions are preferred according to the invention which, for the support of the remineralization process by the poorly water-soluble calcium salt or its composite materials, additionally contain 0.1 to 10% by weight, preferably 0.1 to 5% by weight and in particular, 0.1 to 3% by weight of a remineralization promotion component, in each case based on the total weight of the composition.

The remineralization promotion component assists the remineralization of the enamel and the sealing of dental lesions in the compositions according to the invention and is selected from fluorides, micro-particulate phosphate salts of calcium such as, for example, calcium glycerol phosphate, calcium hydrogenphosphate, hydroxyapatite, fluoroapatite, F-doped hydroxyapatite, dicalcium phosphate dihydrate and calcium fluoride. However, magnesium salts such as, for example, magnesium sulfate, magnesium fluoride or magnesium monofluorophosphate also have a remineralizing action.

Preferred remineralization promotion components according to the invention are magnesium salts.

Suitable embodiments of the oral and dental care composition according to the invention are solid, liquid or semiliquid toothpastes and dental gels.

According to a further preferred embodiment, the oral and dental care compositions according to the invention contain additional toothpaste constituents such as surfactants, humectants, binders, flavorings and active substances against dental and gum diseases.

For improvement of the cleaning action and the foam formation of the oral and dental care compositions according to the invention, surfactants or surfactant mixtures are customarily employed. They promote the rapid and complete dissolution and dispersion of tooth gels in the oral cavity and simultaneously assist mechanical plaque removal, in particular, in the places which are only accessible with difficulty using a toothbrush. The oral and dental care compositions of the invention favor the incorporation of water-insoluble substances, for example, of aromatic oils, stabilize the polishing agent dispersion and assist the anti-caries action of fluorides.

In principle, anionic surfactants, zwitterionic and ampholytic surfactants, nonionic surfactants, cationic surfactants or mixtures of these compounds can be used as surfactants in tooth gel formulations. According to the invention, tooth gels preferably contain at least one surfactant from the group consisting of the anionic surfactants.

The surfactant or the surfactant mixture is customarily employed in the compositions according to the invention in an amount of from 0.1-10% by weight, preferably 0.3-7% by weight and in particular, 1-5% by weight, based on the total weight of the composition.

### Anionic Surfactants.

Suitable surfactants having a good foam action are anionic surfactants, which also have a certain enzyme-inhibiting action on the bacterial metabolism of the plaque.

These include, for example, alkali metal or ammonium salts, in particular, sodium salts, of C₈-C₁₈-alkanecarboxylic acids, of alkyl polyglycol ether sulfates having 12-16 C atoms in the linear alkyl group and 2-6 glycol ether groups in the molecule, of linear alkane-(C₁₂-C₁₈)-sulfonates, sulfosuccinic acid monoalkyl-(C₁₂-C₁₈)-esters, sulfated fatty acid mono-glycerides, sulfated fatty acid alkanolamides, sulfo-acetic acid alkyl-(C₁₂-C₁₆)-esters, acylsarcosines, acyltaurides and acylisethionates in each case having 8-18 C atoms in the acyl group.

The use of at least one anionic surfactant is preferred, in particular, of a sodium alkylsulfate having 12-18 C atoms in the alkyl group. Such a surfactant is sodium laurylsulfate, which is commercially obtainable, for example, under the name Texapon®K12 G.

### Zwitterionic and Ampholytic Surfactants.

It can be preferred according to the invention to employ zwitterionic and/or ampholytic surfactants, preferably in combination with anionic surfactants. Zwitterionic surfactants are designated as those surface-active compounds which carry at least one quaternary ammonium group and at least one carboxylate and one sulfonate group in the molecule. Particularly suitable zwitterionic surfactants are the “betaines” such as the N-alkyl-N,N-dimethylammonium glycinates, for example, trimethylammonium glycinate, coconut alkyl-dimethylammonium glycinate, N-acylaminopropyl-N,N-dimethylammonium glycinates, for example, coconut acyl-aminopropyldimethylammonium glycinate, and 2-alkyl-3-carboxylmethyl-3-hydroxyethylimidazolines in each case having 8 to 18 C atoms in the alkyl or acyl group, and coconut acylaminoethylhydroxyethylcarboxymethyl glycinate. The fatty acid amide derivative known under the CTFA name cocamidopropylbetaine is particularly preferred. Products of this type are commercially obtainable, for example, under the name Tego-betaine®BL 215 and ZF 50 and Genagen®CAB.

Ampholytic surfactants are understood as meaning those surface-active compounds which aside from a C₈-C₁₈-alkyl or acyl group in the molecule contain at least one free amino group and at least one —COOH or —SO₃H group and are capable of the formation of internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropyl-glycines, N-alkyltaurines, N-alkylsarcosines, 2-alkyl-aminopropionic acids and alkylaminoacetic acids in each case having approximately 8 to 18 C atoms in the alkyl group. Particularly preferred ampholytic surfactants are N-coconut alkylaminopropionate, coconut acylamino-ethylaminopropionate and C₁₂-C₁₈-acylsarcosine. In addition to the ampholytic emulsifiers, quaternary emulsifiers are also suitable, those of the type of the ester quats, preferably methyl-quaternized difatty acid triethanolamine ester salts, being particularly preferred.

### Nonionic Surfactants.

Nonionic surfactants are particularly suitable according to the invention for assisting the cleaning action. Particularly preferred nonionic surfactants are those which are selected from at least one of the following groups:
addition products of 2 to 30 mol of ethylene oxide and/or 0 to 5 mol of propylene oxide to linear fatty alcohols having 8 to 22 C atoms, to fatty acids having 12 to 22 C atoms and to alkylphenols having 8 to 15 C atoms in the alkyl group;
C₁₂-C₁₈-fatty acid mono- and diesters of addition products of 1 to 30 mol of ethylene oxide to glycerol;
glycerol mono- and diesters and sorbitan mono- and diesters of saturated and unsaturated fatty acids having 6 to 22 carbon atoms and their ethylene oxide addition products;
alkyl mono- and oligoglycosides having 8 to 22 carbon atoms in the alkyl radical and their ethoxylated analogs;
addition products of 15 to 60 mol of ethylene oxide to castor oil and/or hardened castor oil;
polyol and, in particular, polyglycerol esters, such as, for example, polyglycerol polyricinoleate, polyglycerol poly-12-hydroxystearate or poly-glycerol dimerate.

Mixtures of compounds of a number of these substance classes are likewise suitable:
addition products of 2 to 15 mol of ethylene oxide to castor oil and/or hardened castor oil;
partial esters based on linear, branched, unsaturated or saturated C₆₋₂₂-fatty acids, ricinoleic acid and 12-hydroxystearic acid and glycerol, polyglycerol, pentaerythritol, dipenta-erythritol, sugar alcohols (e.g., sorbitol), sucrose, alkyl glucosides (e.g., methyl glucoside, butyl glucoside, lauryl glucoside) and polyglucosides (e.g., cellulose);
mono-, di- and trialkyl phosphates and mono-, di- and/or tri-PEG-alkyl phosphates and their salts;
wool wax alcohols;
polysiloxane/polyalkyl polyether copolymers and corresponding derivatives;
mixed esters of pentaerythritol, fatty acids, citric acid and fatty alcohol according to DE-C 1165574 and/or mixed esters of fatty acids having 6 to 22 carbon atoms, methylglucose and polyols, preferably glycerol or polyglycerol; and
polyalkylene glycols.

The addition products of ethylene oxide and/or of propylene oxide to fatty alcohols, fatty acids, alkylphenols, glycerol mono- and diesters and sorbitan mono- and diesters of fatty acids or to castor oil are known, commercially obtainable products and are preferred according to the invention. What is involved here is homolog mixtures, whose mean degree of alkoxylation corresponds to the ratio of the amounts of ethylene oxide and/or propylene oxide material and substrate with which the addition reaction is carried out. C₁₂-C₁₈-fatty acid mono- and diesters of addition products of ethylene oxide to glycerol are known from DE-C 2024051 as refatting agents for cosmetic preparations.

C₈-C₁₈-alkyl mono- and oligoglycosides, their preparation and their use are known from the prior art, for example, from U.S. Pat. No. 3,839,318, DE-A 20 36 472, EP-A 77 167 or WO-A 93/10132. Their preparation is carried out, in particular, by reaction of glucose or oligosaccharides with primary alcohols having 8 to 18 C atoms. With respect to the glycoside radical, both monoglycosides, in which a cyclic sugar radical is bonded glycosidically to the fatty alcohol, and oligomeric glycosides having a degree of oligomerization up to preferably approximately 8, are suitable. The degree of oligomerization here is a statistical mean value, which is based on a customary homolog distribution for technical products of this type. Preferably, a suitable alkyl (oligo)glycoside is an alkyl (oligo)glycoside of the formula RO(C₆H₁₀O)ₓ—H, in which R represents an alkyl group having 12 to 14 C atoms and x has a mean value of 1 to 4.

A particularly preferred example of a nonionic surfactant employable according to the invention which may be mentioned is, for example, PEG-glyceryl stearate, which is commercially obtainable under the name Tagat®S.

Humectants are customarily employed in dental cosmetics for protection against drying out and for the regulation of the consistency and cold stability of the products. However, they can also be used for mediating suspension and for influencing taste or luster.

Usually, toxicologically harmless polyols, such as, for example, sorbitol, xylitol, glycerol, mannitol, 1,2-propylene glycol or mixtures thereof are used as humectants, but polyethylene glycols having molecular weights of 400-2,000 can also be used as humectant components in tooth gels.

The combination of a number of humectant components is preferred, where the combination of glycerol and sorbitol with a content of 1,2-propylene glycol or polyethylene glycol is to be regarded as particularly preferred.

Depending on product type, the humectant or the mixture of humectants is contained in the overall composition in an amount of from 10-85% by weight, preferably 15-70% by weight and in particular, 25-50% by weight.

In a preferred embodiment, the compositions according to the invention additionally contain at least one binder or thickener. These have a consistency-regulating effect and furthermore prevent the separation of the liquid and solid constituents.

Their use amounts in the compositions according to the invention are 0.1-5% by weight, preferably 0.1-3% by weight and in particular, 0.5-2% by weight.

According to the invention, for example, natural and/or synthetic water-soluble polymers such as alginates, carrageenans, agar-agar, guar gum, gum arabic, succinoglycan gum, guar flour, carob bean flour, tragacanth, karaya gum, xanthan, pectins, cellulose and their ionic and nonionic derivatives such as, for example, carboxymethylcellulose, hydroxyethyl-cellulose or methylhydroxypropylcellulose, hydrophobically modified celluloses, starch and starch ethers are used.

Water-soluble carboxyvinyl polymers (e.g., Carbopol® types), polyvinyl alcohol, polyvinyl-pyrrolidone and higher molecular weight polyethylene glycols (in particular, those having molecular weights of 10²-10⁶ D), are suitable as binders or thickeners. Likewise, layer silicates and finely divided silicic acids (aerogel silicic acids and pyrogenic silicic acids) can fulfill this function.

In a further preferred embodiment, the oral and dental cleaning composition according to the invention contains additional active substances against dental and gum diseases. Active substances of this type are to be understood as meaning anti-caries active substances, antimicrobial active substances, tartar inhibitors and flavorings according to the invention or any desired combination of these substances.

### Antiplaque Active Substances.

Preferred preparations according to the invention, in particular, oral and dental care compositions and cleaning compositions are characterized in that they additionally comprise anti-plaque active substances, preferably methyl, ethyl or propyl p-hydroxybenzoate, sodium sorbate, sodium benzoate, bromochlorophene, triclosan, phenylsalicylic acid esters, biguanides, e.g., chlorhexidine, thymol, preferably in amounts of from 0.1 to 5% by weight, preferably of from 0.25 to 2.5% by weight and, in particular, of from 0.5 to 1.5% by weight, in each case based on the total composition.

Antimicrobial Active Substances.

Suitable antimicrobial components are, for example, phenols, resorcinols, bisphenols, salicyl-anilides and -amides, and their halogenated derivatives, halogenated carbanilides and p-hydroxy-benzoates.

Among the antimicrobial components, those which inhibit the growth of plaque bacteria are particularly suitable. For example, halogenated diphenyl ethers, such as 2,4-dichloro-2′-hydroxydiphenyl ether, 4,4′-dichloro-2′-hydroxydiphenyl ether, 2,4,4′-tribromo-2′-hydroxy-diphenyl ether, 2,4,4′-trichloro-2′-hydroxydiphenyl ether (triclosan) are suitable as antimicrobial active substances. In addition to bromochlorophene, bisbiguanides such as chlorhexidine and alexidine, phenylsalicylic acid esters and 5-amino-1,3-bis(2-ethylhexyl)hexahydro-5-methylpyrimidine (hexetidine), zinc and copper ions also act antimicrobially, synergistic effects occurring, in particular, in combination with hexetidine and triclosan. Quaternary ammonium compounds, such as, for example, cetyl-pyridinium chloride, benzalkonium chloride, domiphene bromide and dequalinium chloride can also be employed. Octapinol, octenidines and sanguinarin have also proved antimicrobially active.

The antimicrobial active substances are preferably employed in the compositions according to the invention in amounts of from 0.01-1% by weight. Particularly preferably, Irgacare® MP is used in an amount of from 0.01-0.3% by weight.

### Tartar Inhibitors.

Tartar is mineral deposits which are very similar to natural enamel. In order to inhibit tartar formation, substances are added to the dentifrices according to the invention which selectively intervene in crystal seed formation and prevent already present seeds from further growth. These are, for example, condensed phosphates, which are preferably chosen from the group consisting of the tripolyphosphates, of the pyrophosphates, of the trimetaphosphates or their mixtures. They are employed in the form of their alkali metal or ammonium salts, preferably in the form of their sodium or potassium salts. Aqueous solutions of these phosphates typically have an alkaline reaction, so that the pH of the tooth care compositions according to the invention is optionally adjusted to values of 7.5-9 by the addition of acid. Acids which can be used here, for example, are citric acid, phosphoric acid or acidic salts, e.g., NaH₂PO₄. The desired pH of the tooth care compositions, however, can also be adjusted by addition of acidic salts of condensed phosphates, that is, for example, K₂H₂P₂O₇.

Mixtures of various condensed phosphates and/or hydrated salts of the condensed phosphates can also be employed according to the invention. Tartar inhibitors are customarily employed in amounts of from 0.1-5% by weight, preferably 0.1-3% by weight and in particular, 0.1-2% by weight in the compositions according to the invention.

Further suitable tartar inhibitors are organo-phosphonates such as 1-azacycloheptane-2,2-diphos-phonate (Na salt), 1-hydroxyethane-1,1-diphosphonate (Na salt) and zinc citrate.

### Active Substances Against Hypersensitive Teeth.

Preferably, the compositions according to the invention also contain active substances against hypersensitive teeth, which are selected from potassium and strontium salts such as potassium chloride, potassium sulfate, potassium bicarbonate, potassium citrate, potassium acetate, potassium nitrate, strontium chloride, strontium nitrate, strontium citrate, strontium acetate and strontium lactate and eugenol.

The eugenol can be present in the oral and dental care compositions mixed with aromatic oils. Preferably, it is present in the compositions in the form of clove bud oil.

Preferably, the oral and dental care compositions according to the invention contain at least 0.5% by weight of potassium or strontium ions in the form of a dissolved salt and at least 0.01% by weight of eugenol in pure form or in the form of clove bud oil.

### Flavorings.

Preferably, the compositions according to the invention contain flavorings, which include, for example, sweeteners and/or aromatic oils.

Suitable sweeteners are, for example, saccharinates (in particular, sodium saccharinate), cyclamates (in particular, sodium cyclamate) and sucrose, lactose, maltose or fructose.

Possible aromatic oils are all natural and synthetic essences customary for oral and dental care compositions. Natural essences can be used both in the form of the ethereal oils isolated from the drugs (mixture) and in the form of the individual components isolated therefrom. Preferably, at least one aromatic oil from the group consisting of peppermint oil, spearmint oil, aniseed oil, star anise oil, caraway oil, eucalyptus oil, fennel oil, cinnamon oil, oil of cloves, geranium oil, oil of sage, pimento oil, oil of thyme, marjoram oil, basil oil, citrus oil, oil of wintergreen or one/two or more components of these oils isolated therefrom or synthetically produced should be present. The most important components of the oils mentioned are, for example, menthol, carvone, anethole, cineol, eugenol, cinnamaldehyde, caryophyllene, geraniol, citronellol, linalool, salves, thymol, terpenes, terpinol, methylchavicol and methyl salicylate. Further suitable essences are, for example, menthyl acetate, vanillin, ionones, linalyl acetate, rhodinol and piperitone.

Finally, further customary auxiliaries can be present for improving the stability and the sensory properties of the oral and tooth care compositions. Auxiliaries of this type are, for example:
vitamins, e.g., retinol, biotin, tocopherol, ascorbic acid and their derivatives (e.g., esters, salts);
pigments, e.g., titanium dioxide or zinc oxide;
colored pigment particles, for example, colored silicic acid particles, such as are on the market, for example, under the market description Sorbosil® BFG 51, BFG 52 and BFG 53 or Sorbosil®2352. Mixtures of different-colored pigment particles can also be used. Such, for example, strongly orange-, red- or blue-colored gel silicic acid particles can be present in the compositions according to the invention in amounts of from 0.1-1.0% by weight;
bleaching agents such as, for example, hydrogen peroxide and hydrogen peroxide precursors;
colorants;
pH-adjusting agents and buffer substances, e.g., sodium citrate, sodium bicarbonate or potassium and sodium phosphates;
preservatives, e.g., methyl, ethyl or propyl p-hydroxybenzoate, sodium sorbate, sodium benzoate, bromochlorophene or triclosan;
wound-healing and anti-inflammatory substances such as, for example, allantoin, urea, panthenol, azulene or camomile extract, acetylsalicylic acid derivatives, alkali metal thiocyanates; and
mineral salts such as zinc, magnesium and manganese salts, for example, sulfates.

All of these optional toothpaste constituents are present together in the compositions according to the invention in an amount of from approximately 2 to 10% by weight, based on the total weight.

Preparations to be used according to the invention, preferably oral and dental care compositions, in particular, the toothpastes, can also contain substances decreasing the sensitivity of the teeth, for example, potassium salts such as, for example, potassium nitrate, potassium citrate, potassium chloride, potassium bicarbonate and potassium oxalate. Preferred oral and tooth care and cleaning compositions according to the invention are characterized in that they comprise the substances decreasing the sensitivity of the teeth, preferably potassium salts, particularly preferably potassium nitrate and/or potassium citrate and/or potassium chloride and/or potassium bicarbonate and/or potassium oxalate, preferably in amounts of from 0.5 to 20% by weight, particularly preferably of from 1.0 to 15% by weight, further preferably of from 2.5 to 10% by weight and in particular, of from 4.0 to 8.0% by weight, in each case based on the total composition.

Oral and dental care compositions and oral cleaning compositions and dentifrices to be used particularly preferably according to the invention are characterized in that they contain from 0.2 to 20% by weight, preferably 0.4 to 14% by weight, particularly preferably 0.5 to 3% by weight and in particular, 0.6 to 2% by weight, of at least one bioactive glass.

The oral and dental care compositions and oral cleaning compositions and dentifrices of this embodiment to be used according to the invention contain bioactive glass or glass powder or glass ceramic powder or composite materials which comprise a bioactive glass of this type. Glass powders are understood in the context of the present application as also meaning granules and glass beads.

The glass powder should be particularly pure to comply with the requirements that the glass be toxicologically harmless and suitable for consumption. The loading by heavy metals is preferably low. Thus, the maximum concentration in the range of the cosmetic formulations is preferably Pb<20 ppm, Cd<5 ppm, As <5 ppm, Sb<10 ppm, Hg<1 ppm, Ni<10 ppm.

The unceramicized starting glass which is contained directly in the preferred compositions according to the invention, or optionally used for the production of a glass ceramic employable according to the invention, contains SiO₂ as a network former, preferably between 35-80% by weight. At lower concentrations, the spontaneous proneness to crystallization increases greatly and the chemical stability decreases greatly. At higher SiO₂ values, the crystallization stability can decrease, and the processing temperature is markedly increased, such that the heat-shaping properties worsen. Na₂O is employed as a flux when melting the glass. The melting behavior at concentrations of less than 5% is adversely affected. Sodium is a constituent of the phases forming during the ceramicization and must be present, if high crystalline phase contents are to be set by the ceramicization, in correspondingly high concentrations in the glass. K₂O acts as a flux during melting of the glass. In addition, potassium is released in aqueous systems. If high potassium concentrations are present in the glass, potassium-containing phases such as potassium silicates are likewise deposited. By means of the P₂O₅ content, the chemical stability of the glass and thus the ion release in aqueous media can be adjusted in silicate glasses, glass ceramics or composites. In the case of phosphate glasses, P₂O₅ is a network former. The P₂O₅ content is preferably between 0 and 80% by weight. In order to improve the fusibility, the glass can contain up to 25% by weight of B₂O₃. Al₂O₃ is used in order to adjust the chemical stability of the glass. For increasing the antimicrobial, in particular, the antibacterial properties, of the glass ceramic, antimicrobially acting ions such as, for example, Ag, Au, I, Ce, Cu, Zn can be present in concentrations of less than 5% by weight. Color-imparting ions such as, for example, Mn, Cu, Fe, Cr, Co, V can be present individually or combined, preferably in a total concentration of less than 1% by weight.

Customarily, the glass or the glass ceramic is employed in powder form. The ceramicization can be carried out with a glass block, glass ribbons or glass powder. After the ceramicization, the glass ceramic blocks or ribbons must be ground to produce a powder. If the powder has been ceramicized, it must optionally also be ground again in order to remove agglomerates which were formed during the ceramicization step. The grinding can be carried out either dry or in aqueous or nonaqueous grinding media. Customarily, the particle sizes are less than 500 μm. Particle sizes<100 μm or <20 μm have proven to be expedient. Particle sizes<10 μm and also less than 5 μm and also less than 2 μm are particularly suitable. See below.

The bioactive glasses, glass powders, glass ceramic powders or composite compositions contained in the preferred compositions according to the invention comprise glasses which preferably comprise the following components: SiO₂: 35-80% by weight, Na₂O: 0-35% by weight, P₂O₅: 0-80% by weight, MgO: 0-5% by weight, Ag₂O: 0-0.5% by weight, AgI: 0-0.5% by weight, NaI: 0-5% by weight, TiO₂: 0-5% by weight, K₂O: 0-35% by weight, ZnO: 0-10% by weight, Al₂O₃: 0-25% by weight and B₂O₃: 0-25% by weight.

Furthermore, ions such as Fe, Co, Cr, V, Ce, Cu, Mn, Ni, Bi, Sn, Ag, Au and I can be added individually or in total up to 10% by weight to the basic glass according to the above composition for the achievement of further effects such as, for example, colorfulness or UV filtering. A further glass composition can be as follows: SiO₂: 35-80% by weight; Na₂O: 0-35% by weight; P₂O₅: 0-80% by weight; MgO: 0-5% by weight; Ag₂O: 0-0.5% by weight; AgI: 0-0.5% by weight; NaI: 0-5% by weight; TiO₂: 0-5% by weight; K₂O: 0-35% by weight; ZnO: 0-10% by weight; Al₂O₃: 0-25% by weight; B₂O₃: 0-25% by weight; SnO: 0-5% by weight; CeO₂ 0-3% by weight; and Au: 0.001-0.1% by weight.

Particularly preferred oral and tooth care compositions and oral cleaning compositions and dentifrices according to the invention are characterized in that the bioactive glass—based on its weight—has the following composition:

| | | | | | |
|---|---|---|---|---|---|
| SiO₂ | 35 to 60% | by wt, | preferably | 40 to 60% | by wt, |
| Na₂O | 0 to 35% | by wt, | preferably | 5 to 30% | by wt, |
| K₂O | 0 to 35% | by wt, | preferably | 0 to 20% | by wt, |
| P₂O₅ | 0 to 10% | by wt, | preferably | 2 to 10% | by wt, |
| MgO | 0 to 10% | by wt, | preferably | 0 to 5% | by wt, |
| CaO | 0 to 35% | by wt, | preferably | 5 to 30% | by wt, |
| Al₂O₃ | 0 to 25% | by wt, | preferably | 0 to 5% | by wt, |
| B₂O₃ | 0 to 25% | by wt, | preferably | 0 to 5% | by wt, |
| TiO₂ | 0 to 10% | by wt, | preferably | 0.1 to 5% | by wt. |

As already mentioned above, the bioactive glass is preferably employed in particulate form. Oral and dental care compositions and oral cleaning compositions and dentifrices according to the invention particularly preferred here are characterized in that the antimicrobial glass has particle sizes of <10 μm, preferably from 0.5 to 4 μm, particularly preferably from 1 to 2 μm.

A preferred embodiment is the use of toothpastes having a content of silicic acid, preferably precipitation silicic acids, in particular, silicic acids which also contain particles with a size of less than 5 μm, preferably with a size of less than 500 nm, especially preferably with a size of less than 100 nm, polishing agents, humectants, binders and essences, which contain 0.00001 to 10, in particular, 0.01 to 4% by weight, preferably 0.01 to 2% by weight, of the poorly water-soluble calcium salt according to the invention and/or its composite materials, where the calcium salts are selected from the group consisting of hydroxyapatite, fluoroapatite and calcium fluoride.

In particular, when employing the calcium salts used according to the invention and/or their composite material in products for daily oral and dental care, in particular, in toothpaste, it is desirable that the process of remineralization and neomineralization proceeds particularly effectively and rapidly.

A further subject of the present invention relates to candies comprising poorly water-soluble calcium salts according to the invention and/or their composite materials. Preferably, at least 0.000001% by weight of at least one poorly water-soluble calcium salt or its composite material is contained.

According to a further preferred embodiment, the candy is selected from the group consisting of confectionery. Confectionery is a varied group of foods which, according to the guideline for confectionery of the Federal Association of the German Confectionery Industry usually have a distinctively sweet taste because of sugar and/or other commercially customary types of sugar, if desired sugar alcohols, sweeteners or other sweet ingredients. Confectionery is also filling, frosting or candy compositions, and also layers, coatings or fillings of candies or fine baked products. Sugar-free confectionery also ranks as confectionery. In this, the sweet taste is achieved by means of sugar alcohols and/or sweeteners.

Preferred confectionery is, in particular, hard and soft caramels, gumdrops, jelly products, aerated confectionery, licorice products, sugar-coated candy, pastilles and candied fruit.

Caramels (also called candies) in general obtain their originality by boiling down a solution of types of sugar and/or sugar alcohols and are produced in various forms with and without filling using odor- and taste-imparting substances, coloring substances and/or substances influencing the make-up. The make-up of the caramels extends from hard caramels, e.g., drops, to the soft caramels which, in particular, differ by their residual water content. This can be up to approximately 5% by weight in hard caramels and up to approximately 15% by weight in soft caramels. Soft caramels are, for example, the elastic chewing gum-like chewing drops or the soft, readily chewable, in some cases sticky toffees. Candies are differentiated by the manner of their production, for example, cutting, stamping, pouring and laminating.

Jelly products within the sense according to the invention are elastic, soft confectionery with a chewy consistency (e.g., jelly fruit). Likewise, candies according to the invention are gumdrops such as, for example, fruit gums, gummi bears, wine gums or gum pastilles. They are viscoplastic and resistant to chewing. Just like the jelly products, they are produced from types of sugar and/or sugar alcohols, gelling agents (such as agar, pectin or gum arabic), gelatin and/or starch (optionally modified). Waxes or vegetable oils can additionally be employed as mold release and lustering agents.

Licorice products are produced from a mixture of types of sugar and/or sugar alcohols, gelatin and/or (also modified) starch and/or flour and/or gelling agents and thickeners, and various essences. Licorice products contain at least 3% of licorice extract (Succhus liquiritiae; in the commercially customary dry form) as a characteristic ingredient. The addition of up to 8% by weight, in particular, up to 2% by weight, of sal ammoniac leads to “star licorice.”

Sugar-coated candies consist of a smooth or wrinkled covering, produced in the sugar-coating process using types of sugar and/or sugar alcohols, types of chocolate and/or other glazes, and a liquid, soft or solid core. In the sugar-coating process, for example, a saturated sugar solution is sprayed in finely disperse form from a nozzle onto the core rotating in sugar-coating pans. The sugar crystallizes because of the simultaneously blown-in warm air and gradually forms many thin layers around the core. If the sugar layer contains no residual moisture, the candy is designated as a hard sugar-coated candy. In the case of soft sugar-coated candies, on the other hand, approximately 6 to 12, in particular, 8 to 10% by weight of residual moisture can be added. Sugar-coated candies are often provided externally with a thin separating and gloss layer, the gloss layer being formed by treatment with waxy substances, such as, for example, carnauba wax. In particular, substances influencing the make-up, such as, for example, starch and coloring, odor- and flavor-imparting substances are employed.

Compressed tablets or pastilles are produced in a tableting or powder pouring or extrusion process and in addition to the types of sugar and/or sugar alcohols in addition contain, if desired, small amounts of binders and glidants.

According to a particularly preferred embodiment, the candy is a hard or soft caramel or a sugar-coated candy. These candies have the advantage that they are kept in the mouth over a fairly long period and the nanoparticulate calcium salts contained in the candy and/or its composite materials are released only gradually. The mineralizing, and in particular, the neomineralizing, action is thereby particularly promoted.

In particular, in the case of candies made from melts of sugar and/or sugar alcohols, such as, for example, caramels, the active substance can advantageously be incorporated directly into the melt. Surprisingly, a crystallization of the sugar does not occur in the melts here, which on the addition of conventional, ground apatite leads to a polycrystalline mass which is difficult to process. It was likewise not possible to detect the sandy taste occurring with more coarse-grain apatite.

The hard caramels, such as, for example, hard candies, drops, sticks of candy or lollipops, which remain in the mouth for a particularly long time, whereby the gradual release of the active calcium salt or of its composite is optimally afforded, are particularly preferred.

Despite the potentially tooth-damaging ingredients (sugar), the enjoyment of the candy according to the invention, in addition to the primary tasting experience, leads to dental care and dental protection and to the mineralization of the enamel and/or of the dentine. The dental care, customarily with a toothbrush, toothpaste and/or mouthwash, which is not always possible after the eating of candies, but necessary up to now for maintaining the health of the teeth, can thus be omitted without damage to the teeth because of the eating of candies.

According to the invention, preferred sugar alcohols are sorbitol and sorbitol syrup, mannitol, xylitol, lactitol, isomalt, maltitol and maltitol syrup. These substances have the advantage that per 100 g they contain fewer calories and the degradation of the sugar alcohols to acids by some bacteria in the oral cavity takes place so slowly that they do not have a cariogenic action. The addition according to the invention of nanoparticulate calcium salts used according to the invention and/or their composite materials in sugar substitute-containing candies causes mineralization of the teeth during and/or after the eating of the candy and thus particularly contributes to the maintenance of healthy teeth.

According to a further preferred embodiment, the candy according to the invention is filled. Candies having a solid, gelatinous or liquid core make possible, inter alia, the addition of further flavorful components to this core. Likewise, active substances can thereby be introduced which cannot be incorporated directly (for example, by mixing) without a reduction in or loss of action. In hard candies, vitamins or alcohol, inter alia, can be incorporated into fillings of this type.

It is particularly preferred that the filling contains the calcium salt and/or its composite. The calcium salt and/or its composite contained in the filling can thus also be incorporated into those candies in which the danger exists of a loss of action owing to the properties of the candy or the production thereof. This filling can in particular, be a suspension, a gel or a syrup. In particular, the suspensions or gels can be prepared on a water basis in order to guarantee good compatibility. Addition of dispersants or wetting agents suitable for food can be used to keep the calcium salts used according to the invention and/or their composite materials in suspension. Suitable gel-forming agents, in particular, are organic thickeners and their derivatives.

In addition to synthetic organic thickeners, natural organic thickeners, in particular, agar-agar, carrageenans, tragacanth, gum arabic, alginates, pectins, polyoses, guar flour, carob bean flour, starch, dextrins, gelatin and casein are particularly suitable. Natural substances modified therefrom are likewise preferred, in particular, carboxymethyl-cellulose and other cellulose ethers, hydroxyethyl-cellulose and hydroxypropylcellulose, and prime flour ethers. Synthetic organic thickeners such as, for example, polyethers or inorganic thickeners such as polysilicic acids and/or clay minerals (e.g., montmorillonite, zeolites or silicic acids) can likewise be employed according to the invention.

According to a further embodiment of the present invention, the candy is a filled chewing gum.

Chewing gums which contain, according to the invention, poorly water-soluble calcium salts and/or their composite materials incorporated into the chewing composition release only small amounts of the active substance, however, because of their sticky consistency. By biting open the filled chewing gum, poorly water-soluble calcium salt and/or its composite materials, which is contained in the filling, is released directly in the mouth and can thus act better than in conventional chewing gums. The chewing gum additionally promotes the flow of saliva owing to the chewing motion carried out. The caries-causing acids are diluted and thus naturally assist the health of the oral cavity. Chewing gums which have a particularly tooth-caring and -protecting action contain sugar substitutes, in particular, sugar alcohols.

Chewing gums consist of types of sugar and/or sugar alcohols, sweeteners, flavorings, other odor- and taste- or consistency-imparting ingredients, colorants and a water-insoluble chewing composition which becomes plastic on chewing. In addition, the chewing gums can also contain release and coating agents (such as, for example, talc).

Chewing compositions are mixtures of consistency-imparting substances, the natural gums, that is solidified juices (exudates) of tropical plants such as chicle, gum arabic, gutta-percha, karaya gum and tragacanth, rubber and the thermoplastic synthetic substances butadiene/styrene copolymers, isobutylene/isoprene copolymers, polyethylene, polyisobutylene, polyvinyl esters of the unbranched fatty acids from C₂ to C₁₈ and polyvinyl ethers.

As plasticizers, resins and balsams are employed. The natural substances are benzoin resin, dammar resin, colophony, mastic, myrrh, olibanum, Peru balsam, sandarac, shellac and Tolu balsam, the synthetic substances are coumarone indene resin, glycerol pentaerythritol esters of the resin acids of colophony and of their hydrogenation products.

For influencing the elasticity, paraffins (natural and synthetic) and waxes are used. The waxes are those from the plant field such as carnauba wax and those of animal origin such as beeswax or wool wax. In addition those from the mineral field such as microcrystalline waxes, and also chemically modified or synthetic waxes are used. As plasticizers, emulsifiers are used (e.g., lecithins or mono- and diglycerides of food fatty acids) and esters such as glycerol acetate and also glycerol.

For the regulation of the chewing composition consistency, plant hydrocolloids such as agar-agar, alginic acid and alginates, prime guar flour, carob bean flour or pectin are added. For the selected adjustment of the chewing properties of chewing compositions, fillers are employed. These are carbonates of calcium or magnesium, oxides, for example, alumina, silicic acid and silicates of calcium or magnesium. Stearic acid and its calcium and magnesium salts are employed for the reduction of the adhesive power of the chewing composition to the enamel.

Before the remaining ingredients according to the recipe necessary for the production of chewing gum are intermixed, it is necessary to heat the chewing composition, which makes up approximately 20-35% (but at least 15%) of the finished chewing gum, to 50-60° C.

According to a particularly preferred embodiment of the present invention, the chewing gum is covered by at least one layer, this layer comprising at least one calcium composite material according to the invention.

For the production of chewing gums according to the invention, the composite materials are simply added to the chewing composition. Alternatively, a coated chewing gum can also be prepared according to the invention, in which the covering layer contains the two essential ingredients of the products according to the invention. For the production of coated chewing gums according to the invention of this type, the active substances, that is the calcium salt and/or its composite, are simply added to a solution and/or dispersion from which the covering is produced, and stirred in.

In a preferred embodiment of the present invention, the chewing gum according to the invention is a sugar-containing chewing gum. In connection with the present invention, “sugar” and “types of sugar” are understood as meaning products such as sucrose, purified crystalline sucrose, for example, in the form of refined sugar, refined products, refined white sugar, white sugar or semi white sugar, aqueous solutions of sucrose, for example, in the form of liquid sugar, aqueous solutions of sucrose partially inverted by hydrolysis, for example, invert sugar, syrup or invert liquid sugar, glucose syrup, dried glucose syrup, water of crystallization-containing dextrose, water of crystallization-free dextrose and other starch saccharification products and also trehalose, trehalulose, tagatose, lactose, maltose, fructose, leucrose, isomaltulose (palatinose), condensed palatinose and hydrogenated condensed palatinose. The sugar-containing chewing gum according to the invention is, therefore, characterized in that either the chewing gum itself or the covering layer or both contain sucrose, invert liquid sugar, invert sugar syrup, glucose, glucose syrup, polydextrose, trehalose, trehalulose, tagatose, lactose, maltose, fructose, leucrose, isomaltulose (palatinose), condensed palatinose, hydrogenated condensed palatinose or mixtures thereof as sweetening agents. In addition to the above-mentioned types of sugar, the sugar-containing chewing gum according to the invention can also contain sugar substitutes, in particular, sugar alcohols such as lactitol, sorbitol, xylitol, mannitol, maltitol, erythritol, 6-O-α-D-glucopyranosyl-D-sorbitol (1,6-GPS), 1-O-α-D-glucopyranosyl-D-sorbitol (1,1-GPS), 1-O-α-D-glucopyranosyl-D-mannitol (1,1-GPM), maltitol syrup, sorbitol syrup, fructooligosaccharides or mixtures thereof, and mixtures of sugar alcohols and types of sugar.

In a further preferred embodiment of the invention, the chewing gum according to the invention is a sugar-free chewing gum. In connection with the present invention, a “sugar-free chewing gum” is understood as meaning a chewing gum in which both the chewing gum itself and the covering layer contain none of the above-mentioned types of sugar as sweetening agent, that is neither sucrose, invert liquid sugar, invert sugar syrup, dextrose, glucose syrup, trehalose, trehalulose, tagatose, lactose, maltose, fructose, leucrose, isomaltose (palatinose), condensed palatinose, hydrogenated condensed palatinose nor mixtures thereof, but rather sugar substitutes. In a preferred embodiment of the invention, the sugar-free chewing gum according to the invention is a chewing gum which contains a maximum content of the above-mentioned types of sugar of 0.5% by weight, based on the dry weight.

The term “sugar substitutes” comprises all substances, aside from the above-mentioned types of sugar, which can be used for the sweetening of foods. The term “sugar substitutes” in particular, comprises substances such as hydrogenated mono- and disaccharide sugar alcohols, for example, lactitol, xylitol, sorbitol, mannitol, maltitol, erythritol, isomalt, 1,6-GPS, 1,1-GPS, 1,1-GPM, sorbitol syrup, maltitol syrup, and fructooligosaccharides. Preferably, the sugar-free chewing gums according to the invention are thus characterized in that both the chewing gum itself and the covering layer contain lactose, maltose, fructose, leucrose, palatinose, condensed palatinose, hydrogenated condensed palatinose, fructooligo-saccharides, lactitol, sorbitol, xylitol, maltitol, erythritol, 1,6-GPS, 1,1-GPS, 1,1-GPM, sorbitol syrup, maltitol syrup or mixtures thereof as sweetening agent. According to the invention, sugar alcohols such as sorbitol and sorbitol syrup, mannitol, xylitol, lactitol, maltitol and maltitol syrup, 1,1-GPS, 1,6-GPS, 1,1-GPM or mixtures thereof are preferred. Sugar alcohols have the advantages that per 100 g they contain fewer calories and are only degraded to acids very slowly or not at all by bacteria of the oral flora. Thus, they have no cariogenic action.

A preferably used mixture of 1,6-GPS and 1,1-GPM is isomalt, in which 1,6-GPS and 1,1-GPM are present in equimolar or nearly equimolar amounts. According to the invention, 1,6-GPS-enriched mixtures of 1,6-GPS and 1,1-GPM having a 1,6-GPS content of 57% by weight to 99% by weight and a 1,1-GPM content of 43% by weight to 1% by weight, 1,1-GPM-enriched mixtures of 1,6-GPS and 1,1-GPM having a 1,6-GPS content of 1% by weight to 43% by weight and a 1,1-GPM content of 57% by weight to 99% by weight, and mixtures of 1,6-GPS, 1,1-GPS and 1,1-GPM can likewise be employed as sweetening agents in the chewing gums according to the invention, in particular, sugar-free chewing gums, both in the chewing gum itself and in the layer covering it. 1,6-GPS-enriched mixtures and 1,1-GPM-enriched mixtures of 1,6-GPS and 1,1-GPM are disclosed in DE 195 32 396 C2, the disclosure content of this publication with respect to the description and preparation of the 1,6-GPS-enriched and 1,1-GPM-enriched sweetening agent mixtures being completely additionally included in the disclosure content of the present teaching. 1,1-GPS-containing mixtures of 1,6-GPS and 1,1-GPM are disclosed for example, in EP 0 625 578 B1, the disclosure content of this publication with respect to the description and preparation of the 1,1-GPS-, 1,6-GPS- and 1,1-GPM-containing sweetening agent mixtures also being completely included in the disclosure content of the present teaching.

A further preferred mixture according to the invention which can be employed in the chewing gums according to the invention, in particular, sugar-free chewing gums, is a syrup with a dry matter content of 60 to 80%, consisting of a mixture of hydrogenated starch hydrolyzate syrup and isomalt powder or isomalt syrup, where the dry substance of the syrup of 7 to 52% (weight/weight) of 1,6-GPS, 24.5 to 52% (weight/weight) of 1,1-GPM, 0 to 52% (weight/weight) of 1,1-GPS, 0 to 1.3% (weight/weight) of sorbitol, 2.8 to 13.8% (weight/weight) of maltitol, 1.5 to 4.2% (weight/weight) of maltotriitol and 3.0 to 13.5% (weight/weight) of higher polyols. Such a syrup is disclosed in EP 1 194 042 B1, the disclosure content of this publication with respect to the description and preparation of the syrup consisting of a mixture of hydrogenated starch hydrolyzate syrup and isomalt powder or isomalt syrup also being completely included in the disclosure content of the present teaching.

The sugar-free chewing gum according to the invention, which is covered by at least one layer which comprises composite material according to the invention, can be, for example, a hard-coated sugar-free chewing gum which comprises a sugar-free chewing gum core and a sugar-free hard coating which contains an essentially hygroscopic sugar-free sweetening agent, the chewing gum core having a water content of less than approximately 2.5% by weight, based on the weight of the core. The essentially hygroscopic sweetening agents can be, for example, sorbitol or hydrogenated isomaltulose. Such sugar-free hard-coated chewing gums are described in WO 88/08671, the disclosure content of this publication with respect to the description and preparation of the hard-coated sugar-free chewing gums also being completely included in the disclosure content of the present teaching.

In a further embodiment of the invention, it is provided for both the sugar-containing chewing gums according to the invention and the sugar-free chewing gums according to the invention additionally to be able to contain one or more intensive sweeteners in the chewing gum itself and/or in the layer covering it, in addition to the above-mentioned types of sugar and/or sugar substitutes. Intensive sweeteners are compounds which, combined with a small or negligently small nutrient value, are distinguished by an intensive sweet taste. According to the invention, it is, in particular, provided that the intensive sweetener employed in the chewing gums according to the invention is cyclamate, for example, sodium cyclamate, saccharin, for example, saccharin sodium, Aspartame®, glycyrrhizin, neohesperidin dihydrochalcone, thaumatine, monellin, acesulfam, stevioside, alitam, sucralose or a mixture thereof. Using intensive sweeteners of this type, the content of types of sugar can, in particular, be reduced, and in spite of this, the predominantly sweet taste can be obtained.

In a further embodiment of the invention, it is proposed that the chewing gum according to the invention not only has a covering layer, in particular, a sugar-coated layer, which comprises a poorly soluble calcium salt and/or its composites, but at least 2 to approximately 100 covering layers of this type, in particular, sugar-coated layers. According to the invention, it is possible that the individual layers contain the same sweetening agent or the same sweetening agents. Of course, according to the invention the possibility also exists that the individual layers can also contain different sweetening agents. Such sugar-coated chewing gum products are thus covered by layer sequences of different sweetening agent compositions. By means of a suitable choice of the sequence and number of the coating steps with the different sweetening agents, the production of specific chewing gums with desired properties is possible.

For example, the chewing gum according to the invention can first be covered with 1 to approximately 45 sugar-coated layers, which contain the 1,1-GPM-enriched mixture of 1,6-GPS and 1,1-GPM. Subsequently, 1 to approximately 45 layers of the 1,6-GPS-enriched mixture of 1,6-GPS and 1,1-GPM are applied to these layers. Such a sugar-coated chewing gum is distinguished because of the higher solubility and greater sweetening power of the 1,6-GPS-enriched mixture forming the outer layer by an altogether higher sweetening power in comparison to, for example, chewing gums coated with hydrogenated isomaltulose. Such a layer sequence is described in DE 195 32 396 C2, the disclosure content of this publication with respect to the description and preparation of chewing gums having this layer sequence also being completely included in the disclosure content of the present teaching.

For example, the chewing gum according to the invention can be a hard-coated chewing gum, the sugar-coated candy covering containing a number of layers which contain approximately 50% to approximately 100% xylitol, and a number of layers which contain approximately 50% to approximately 100% hydrogenated isomaltulose. Such chewing gums are disclosed in WO 93/18663, the disclosure content of this publication with respect to the description and preparation of chewing gums having this layer sequence also being completely included in the disclosure content of the present teaching.

In a further embodiment, it is proposed that the individual sugar-coated layers covering the chewing gum contain the same calcium salt and/or the same composites thereof. According to the invention, it is also possible, however, that the individual layers which cover the chewing gum contain different calcium salts and/or different composites thereof. Of course, the possibility also exists that individual layers contain no calcium salt or no composites thereof.

The layer covering the chewing gum, which comprises the poorly water-soluble calcium salt, advantageously leads to the release of the calcium salt taking place more simply than in the case of the direct incorporation of the salts into the chewing gum composition, in which the incorporated calcium salts remain strongly attached to the sticky matrix of the chewing composition. The layer covering the chewing gum dissolves very quickly on chewing in the mouth. The necessary amount of active substance can thus be made available in the mouth, advantageously guaranteeing an effective mineralization of the teeth. By means of the addition of the calcium salts and/or composites thereof, the crunch, that is the crispness of the chewing gum, is not influenced.

According to a particular embodiment, the layer covering the chewing gum according to the invention contains sugar and/or sugar alcohols. The types of sugar preferably to be used here are mono-, di- and oligosaccharides such as, for example, dextrose, fructose and sucrose, glucose syrup, liquid sugars and related products, dried glucose syrup and other starch saccharification products and also sugar substitutes, in particular, sugar alcohols.

Advantageously, the layer containing the sugars and/or sugar alcohols dissolves particularly rapidly in the mouth. In addition to providing the sweet taste experience, the layer can also be applied particularly readily to a chewing gum core.

Despite the potentially tooth-damaging ingredients (sugars), the enjoyment of the chewing gums according to the invention, in addition to the primary tasting experience, leads to dental care and dental protection and to the mineralization of the enamel and/or of the dentine. The dental care, which is not always possible after eating, but necessary up to now for the maintenance of the health of the teeth, customarily using a toothbrush, toothpaste and/or mouthwash, can thus be omitted without damage to the teeth.

Sugar alcohols preferred according to the invention are sorbitol and sorbitol syrup, mannitol, xylitol, lactitol, isomalt, maltitol and maltitol syrup. These substances have the advantage that per 100 g they contain fewer calories and, in addition, the degradation of the sugar alcohols to acids by some bacteria of the oral cavity takes place so slowly that they do not have a cariogenic action. The addition according to the invention of composites according to the invention in sugar substitute-containing chewing gums causes a mineralization of the teeth during and/or after the partaking of the chewing gum and thus contributes particularly to the maintenance of healthy teeth. Advantageously, the sugar alcohols are particularly suitable because of their physicochemical properties for the production of thin layers, especially in the sugar-coating process. The use of isomalt in the covering layer is particularly preferred, as this sugar alcohol has a comparatively high glass transition temperature, which particularly facilitates processing.

The layer covering the chewing gum can be produced in different ways, e.g., by repeated dipping of the chewing gum core into an appropriate solution and/or dispersion.

According to a preferred embodiment of the invention, at least one layer covering the chewing gum is a sugar-coated layer, i.e., the layer is applied to the chewing gum in the sugar-coating process. The sugar-coated layer (covering) may be smooth or wrinkled, depending on the type of sugar and/or sugar alcohol, type of chocolate and/or other glaze, which is applied around a liquid, soft or solid core by means of the sugar-coating process (as described above).

According to a further embodiment, the chewing gum is a filled chewing gum.

Chewing gums which contain composite materials according to the invention incorporated into the chewing composition, however, release only small amounts of the active substance because of their sticky consistency. As a result of biting open the filled chewing gum, the composite material according to the invention, which is contained in the filling, is released directly in the mouth and can thus act better than in conventional chewing gums.

The filled chewing gum can also contain at least one layer covering the chewing gum comprising the composite materials according to the invention.

According to a further preferred embodiment of the present invention, the candy comprises a dissolving component. This component or matrix dissolves in the mouth by contact with the saliva. The dissolution can also be achieved here by having the candy remain in the mouth longer (in particular, of over five minutes) and/or sucking. A component or matrix is to be understood here, for example, as meaning the sugar matrix or basic composition of a hard candy, of a gumdrop or alternatively a filling.

It is particularly preferred that the poorly water-soluble calcium salts and/or their composite materials contained according to the invention in the candy are situated in the dissolving component or matrix. This advantageously leads to the dissolving component being able to release the active substance situated or contained therein in the mouth. In particular, this is important for those candies in which the active substance is otherwise not released in larger amounts.

This can be advantageous, for example, in a filled chewing gum. The poorly water-soluble calcium salts used according to the invention and/or their composites are incorporated into a solid, gelatinous or liquid filling which, as a result of biting open the chewing gum in the mouth, exits from the chewing gum and releases the active substance. In the case of a liquid filling, this mixes with the saliva. It is also possible that the calcium salt and/or its composite, for example, is incorporated into a chewing gum in granulated sugar beads. It is likewise possible that the calcium salt and/or its composites is applied to the candy as finely powdered dust, for example, together with release agents for chewing gums (e.g., with talcum) or acid drops (which are often dusted, for example, with powdered sugar as protection against sticking together).

The active substance situated in the dissolving component or matrix does not remain, as in the incorporation into the chewing composition of a chewing gum, to a large extent attached to or with a non-dissolving component. Thus, the necessary amount of active substance is made available in the mouth, which advantageously guarantees effective mineralization of the teeth.

According to a particularly preferred embodiment of the invention, the candy consists essentially of at least one dissolving component or matrix. According to the invention, it is particularly advantageous here that there is no component of the candy to which the active substance can furthermore be bound in the mouth after sucking or dissolving and can thus be present in a form not available for the mineralization of the dental material. Appropriate candies can be, for example, filled or unfilled caramels, gumdrops, jelly products, aerated confectionery, licorice products, sugar-coated candies or pastilles.

According to a further embodiment, the candy contains flavorings, sweeteners, fillers and/or other auxiliaries (such as, for example, glycerol or mineral salts, for example, Zn²⁺ or Mg²⁺).

Any natural or naturally identical aromatic substances, such as, for example, fruit essences, can be employed. These can, in particular, be present in the solid or liquid fruit preparations, fruit extracts or fruit powders. Pineapple, apple, apricot, banana, blackberry, strawberry, grapefruit, blueberry, raspberry, maracuja, orange, sour cherry, red- and blackcurrant, woodruff and lemon are preferred here.

Other essences, in particular, aromatic oils such as, for example, peppermint oil, spearmint oil, eucalyptus oil, aniseed oil, fennel oil, caraway oil and synthetic aromatic oils can also be employed. This occurs particularly preferably in herbal drops and/or cough drops and chewing gums.

Further taste-imparting additives can be, inter alia: milk, yoghurt, cream, butter, honey, malt, caramel, licorice, wine, almond, pistachio, hazelnut or walnut kernels and other protein-rich oilseeds and peanut kernels, coconut, cocoa, chocolate, cola or vanilla.

Active substances, such as, for example, menthol and/or vitamins, can also be present in the candy according to the invention. Likewise, organophosphonates, such as, for example, 1-hydroxy-ethane-1,1-diphosphonic acid, phosphonopropane-1,2,3-tricarboxylic acid (Na salt) or 1-azacycloheptane-2,2-diphosphonic acid (Na salt), and/or pyrophosphates can be added, which prevent the formation of tartar.

Sweetening agents such as, for example, saccharin sodium, acesulfam K, Aspartame®, sodium cyclamate, stevioside, thaumatin, sucrose, lactose, maltose, fructose or glycyrrhizin are likewise preferably present. The content of sugar can thus be reduced and yet the predominantly sweet taste can be maintained.

Preservatives which can be employed are all preservatives permitted for food, for example, sorbic or benzoic acid and their derivatives, such as, for example, sodium benzoate and parahydroxybenzoate (sodium salt), sulfur dioxide or sulfurous acid, sodium nitrite or potassium nitrite. Colorants and pigments for achieving an attractive appearance can likewise be present.

A further subject of the present invention is the use of at least one poorly water-soluble calcium salt according to the invention and/or its composite materials in the candies, in particular, confectionery, as an ingredient having a positive action on dental health.

In particular, the poorly water-soluble calcium salts used according to the invention and/or their composite materials or the candy containing them are employed for dental care and dental protection and for the mineralization of the enamel and/or of the dentine. Carious disease of the teeth can thus be counteracted by the use according to the invention of poorly water-soluble calcium salts and/or their composite materials. In addition to the satisfaction of consumption, the candy according to the invention can thus additionally be used for caries prophylaxis.

According to a further preferred embodiment, the compositions to be used according to the invention additionally contain at least one fluoride salt in addition to the poorly water-soluble calcium salt and/or its composite materials.

Preparations used according to the invention, and, in particular, oral and dental care compositions and oral cleaning compositions and dentifrices, which additionally contains anticaries active substances, preferably fluoride compound(s), in particular, sodium fluoride, potassium fluoride, sodium monofluoro-phosphate, zinc fluoride, tin fluoride and sodium fluorosilicate, preferably in amounts of from 0.01 to 5% by weight, preferably of from 0.1 to 2.5% by weight and in particular, of from 0.2 to 1.1% by weight.

It is known from the prior art that formulations comprising poorly water-soluble calcium salts, in particular, apatites, and free fluoride (for example, as the alkali metal or ammonium salt) show no effects on the hardening of the enamel because the apatite binds that contained in the formulation by exchange processes during preparation and storage, and thus the fluoride in the formulation is no longer present in free form.

Surprisingly, it has been found that the addition of fluoride leads to a synergistic increase in the nucleating effect of the calcium salts used according to the invention and/or their composite materials. The addition of sodium fluoride and/or potassium fluoride is particularly preferred. On simultaneous addition of calcium salts used according to the invention and/or their composite materials and small amounts of fluoride, an approximately five-fold synergistic increase is seen. According to the invention, amounts of from 0.01 to 1.2% by weight, in particular, from 0.1 to 0.90% by weight, of fluoride salt are preferred, depending on the fluoride salt used (e.g., sodium fluoride). This corresponds to an amount of fluoride employed of from 0.05 to 0.15% by weight, in particular, of from 0.08 to 0.12% by weight.

According to the invention, amounts of from 0.05 to 0.15% by weight, in particular, of from 0.08 to 0.12% by weight, of fluoride are preferred, based on the amount of fluoride ions.

The fluoride is, in particular, contained in the formulations to <2500 ppm, preferably between 10 and 1500 ppm of fluoride. The fluoride can be added, for example, in the form of alkali metal or ammonium fluorides. A molar ratio between the fluoride employed and the calcium salt employed, in particular, hydroxyapatite, of greater than one, particularly preferably of greater than three, is particularly favorable.

Surprisingly, it has been found that formulations of this type have an optimal suitability for the uses according to the invention and exert an additionally hardening influence on the bone and dental material, in particular, the enamel, without binding the fluoride in the formulation by means of the added poorly water-soluble calcium salt and/or its composite materials and thus inactivating it for use for hardening the teeth.

A very particularly preferred embodiment is the use of toothpastes containing silicic acid, preferably silicic acids having a particle size of less than 5 μm, polishing agents, humectants, binders and essences which contain 0.00001 to 10, in particular, 0.05 to 5% by weight, preferably 0.01 to 2% by weight, of a composite material consisting of poorly water-soluble calcium salt in combination with a polymer component, preferably a protein component, particularly preferably a gelatin, very particularly of the type AB (acid bone), the nanoparticulate calcium salts being selected from the group consisting of hydroxyapatite, fluoroapatite and calcium fluoride. Particularly preferably, fluoride is also contained in these toothpastes, preferably to <2500 ppm, particularly preferably between 10 and 1500 ppm of fluoride. According to the invention, amounts of fluoride of from 0.05 to 0.15% by weight, in particular, of from 0.08 to 0.12% by weight, are preferred.

It has been found that during and after the use of the compositions according to the invention, in particular, in the form of a toothpaste, a particularly smooth, good and, as perceived by test subjects, clean oral sensation results.

The following examples are intended to explain the subject of the invention in greater detail.

### EXAMPLES

### 1. Production of Composite Materials by Precipitation Reactions in the Presence of the Polymer Components

### 1.1 Production of an Apatite-Protein Composite

For the production of the apatite-gelatin composite, 2,000 ml of deionized water are introduced into a 4 l beaker thermostated at 25° C., in which are dissolved 44.10 g (0.30 mol) of CaCl₂.2H₂O (Fisher Chemicals p.a.). Separately from this, 35 g of gelatin (type AB, DGF-Stoess, Eberbach) are dissolved in 350 ml of deionized water at approximately 50° C. The two solutions are combined and vigorously stirred with a propeller stirrer. The pH is adjusted to 7.0 using dilute aqueous base.

300 ml of a 0.6 M (NH₄)₂HPO₄ solution, which had been adjusted to pH 7.0 beforehand, are uniformly pumped into this gelatin and calcium salt solution with vigorous stirring in the course of 120 min using an automated addition apparatus. In the course of this, the pH is kept constant at pH 7.0 by the controlled addition of dilute aqueous base. After completion of the addition, the mixture is stirred further for 24 h.

Subsequently, the dispersion is filled into centrifuge beakers and the solids content is separated by centrifuging the solution. By extracting the residue by shaking in deionized water five times and subsequent fresh centrifugation, the salts are largely washed out, such that chloride is no longer detectable.

The dry composite material contains 43% by weight of organic, i.e., protein, components. This fraction is determined by ashing the material at 800° C. for 3 h or else by the expert analysis of a thermogravimetric measurement or by carbon combustion analysis (CHN) or by Kjeldahl nitrogen analysis, where in each case the proportion of the ammonium chloride contamination is not to be included.

### 2. Production of Composite Materials by Incorporation of Dispersions of Surface-Modified Calcium Salts in Protein Components

### 2.1 Composite Material of Hydroxyapatite and Gelatin AB

First, the solutions A and B were prepared separately.

### Solution A.

25.4 g of calcium nitrate tetrahydrate and 8.50 g of diammonium hydrogenphosphate were in each case dissolved in 100 g of deionized water. The two solutions were mixed together with formation of a white precipitate. After addition of 10 ml of 37% strength by weight HCl, a clear solution was obtained.

### Solution B.

200 ml of deionized water, 200 ml of 25% strength by weight aqueous ammonia solution and 20 g of Plantacare® 1,200 were mixed together and cooled to 0° C. in an ice bath. Solution A was added to solution B with vigorous stirring with formation of a hydroxyapatite precipitate. After stripping off the excess ammonia, the dispersion was purified by means of dialysis. The dispersion was subsequently concentrated on a rotary evaporator by determination of the deposited amount of water to the point where the solids content in the dispersion, calculated as hydroxyapatite, was 7.5% by weight.

This dispersion was added at room temperature to 100 ml of a 10% strength by weight aqueous solution of gelatin type AB (manufacturer: DGF Stoess) prepared analogously to Example 1.1, then heated to 80° C. and stirred at this temperature for 5 minutes. Subsequently, the mass was allowed to solidify at room temperature with formation of the composite material.

### 3. Tooth Gels Containing Calcium Salt Composite Materials

TABLE 1

| | Formulation examples | | | | | | | |
|---|---|---|---|---|---|---|---|---|
| | 3.1 | | 3.2 | | 3.3 | | 3.4 | |
| Sident ® 8 | 10.0% | by wt | 10.0% | by wt | 10.0% | by wt | 10.0% | by wt |
| Sident ® 22S | 7.0% | by wt | 7.0% | by wt | 7.0% | by wt | 7.0% | by wt |
| Sipernat ® 320DS | 0.8% | by wt | 0.8% | by wt | 0.8% | by wt | 0.8% | by wt |
| Glycerol solution | 1.0% | by wt | 0.1% | by wt | 1.0% | by wt | 0.1% | by wt |
| comprising 10% by | | | | | | | | |
| weight of poorly water- | | | | | | | | |
| soluble | | | | | | | | |
| hydroxyapatite | | | | | | | | |
| (nanoparticulate, | | | | | | | | |
| particle diameter less | | | | | | | | |
| than 300 nm | | | | | | | | |
| Polywachs ® 1550 | 2.0% | by wt | 2.0% | by wt | 2.0% | by wt | 2.0% | by wt |
| Texapon ® K 1296 | 1.5% | by wt | 1.5% | by wt | 1.5% | by wt | 1.5% | by wt |
| Titanium dioxide | 1.0% | by wt | 1.0% | by wt | 1.0% | by wt | 1.0% | by wt |
| Cekol ® 500 T | 1.0% | by wt | 1.0% | by wt | 1.0% | by wt | 1.0% | by wt |
| Na fluoride | 0.33% | by wt | 0.33% | by wt | 0.33% | by wt | 0.33% | by wt |
| Na benzoate | 0.25% | by wt | 0.25% | by wt | 0.25% | by wt | 0.25% | by wt |
| Essence | 1.0% | by wt | 1.0% | by wt | 1.0% | by wt | 1.0% | by wt |
| Tagat ® S | 0.2% | by wt | — | | — | | 0.2% | by wt |
| Na saccharinate | 0.15% | by wt | 0.15% | by wt | 0.15% | by wt | 0.15% | by wt |
| Trisodium phosphate | 0.10% | by wt | 0.10% | by wt | 0.10% | by wt | 0.10% | by wt |
| Sorbitol (70% strength | 31.0% | by wt | 31.0% | by wt | 31.0% | by wt | 31.0% | by wt |
| in water) | | | | | | | | |
| Water | To 100% | by wt | To 100% | by wt | To 100% | by wt | To 100% | by wt |

### 3. Tooth Gels Containing Calcium Salt Composite Materials

TABLE 2

| | Formulation examples | | | | | | | |
|---|---|---|---|---|---|---|---|---|
| | 4.1 | | 4.2 | | 4.3 | | 4.4 | |
| Sident ® 8 | 10.0% | by wt | 10.0% | by wt | 10.0% | by wt | 10.0% | by wt |
| Sident ® 22S | 7.0% | by wt | 7.0% | by wt | 7.0% | by wt | 7.0% | by wt |
| Sipernat ® 320DS | 0.8% | by wt | 0.8% | by wt | 0.8% | by wt | 0.8% | by wt |
| Glycerol solution | 1.0% | by wt | 0.1% | by wt | 1.0% | by wt | 0.1% | by wt |
| comprising 10% by | | | | | | | | |
| weight of composite | | | | | | | | |
| material | | | | | | | | |
| Polywachs ® 1550 | 2.0% | by wt | 2.0% | by wt | 2.0% | by wt | 2.0% | by wt |
| Texapon ® K 1296 | 1.5% | by wt | 1.5% | by wt | 1.5% | by wt | 1.5% | by wt |
| Titanium dioxide | 1.0% | by wt | 1.0% | by wt | 1.0% | by wt | 1.0% | by wt |
| Cekol ® 500 T | 1.0% | by wt | 1.0% | by wt | 1.0% | by wt | 1.0% | by wt |
| Na fluoride | 0.33% | by wt | 0.33% | by wt | 0.33% | by wt | 0.33% | by wt |
| Na benzoate | 0.25% | by wt | 0.25% | by wt | 0.25% | by wt | 0.25% | by wt |
| Essence | 1.0% | by wt | 1.0% | by wt | 1.0% | by wt | 1.0% | by wt |
| Tagat ® S | 0.2% | by wt | — | | — | | 0.2% | by wt |
| Na saccharinate | 0.15% | by wt | 0.15% | by wt | 0.15% | by wt | 0.15% | by wt |
| Trisodium phosphate | 0.10% | by wt | 0.10% | by wt | 0.10% | by wt | 0.10% | by wt |
| Sorbitol (70% strength | 31.0% | by wt | 31.0% | by wt | 31.0% | by wt | 31.0% | by wt |
| in water) | | | | | | | | |
| Water | To 100% | by wt | To 100% | by wt | To 100% | by wt | To 100% | by wt |

The following commercial products were used:

| | |
|---|---|
| Plantacare ® 1,200: | C₁₂-C₁₆-alkyl glycoside |
| | about 50% by weight in water |
| | Manufacturer: Cognis |
| Sident ® 8: | synth. amorph. silicic acid, |
| | BET 60 m²/g |
| | Bulk density: 350 g/l |
| | Manufacturer: DEGUSSA |
| Sident ® 22S: | hydrogel silicic acid, |
| | BET 140 m²/g |
| | Bulk density: 100 g/l |
| | Manufacturer: DEGUSSA |
| Polywachs ® 1550: | polyethylene glycol, MW: 1550 |
| | softening point 45-50° C. |
| | Manufacturer: RWE/DEA |
| Texapon ® K 1296: | sodium lauryl sulfate powder |
| | Manufacturer: Cognis |
| Cekol ® 500 T: | sodium carboxymethylcellulose |
| | viscosity (2% in water, Brookfield |
| | LVF, 20° C.): 350-700 mPa · s |
| | supplier: Noviant |
| Tagat ® S | polyoxyethylene-(20)-glyceryl monostearate |
| | Manufacturer: Tego Cosmetics (Goldschmidt) |

### 4. Stability of Fluoride and Apatite in Standard Formulations

As a standard formulation, a gel based on 0.5% CMC, 24% glycerol and 1% apatite-protein composite with a 60% apatite content was prepared.

The gel was divided into two samples. 900 ppm of fluoride were added to Sample 1 and 1500 ppm of fluoride were added to Sample 2 in the form of sodium fluoride. The fluoride content of the gels was determined by ion chromatography immediately after preparation and after 2 and 4 weeks.

As is evident from Table 3, the concentration of free fluoride is constant and at the level employed. Surprisingly, no decrease in the free fluoride content thus takes place.

TABLE 3

| | Sample 1 | Sample 2 |
|---|---|---|
| Week | ppm of F | ppm of F |
| 0 | 895 | 1500 |
| 2 | 930 | 1600 |
| 4 | 865 | 1450 |

5. Stability of the nucleating action—pH measurements in artifical saliva:

### 5.1 Method.

The dispersion which contains the composite materials according to the invention is added to a salt solution whose content of inorganic salts corresponds to that of body fluids such as saliva, blood or plasma (simulated body fluid, SBF) and which is accordingly supersaturated based on the precipitation of calcium phosphate. Compositions of this type can be employed as a model of body fluids, as has already been described in Liu et al., Cells and Materials (1997), 7, pp. 41-51).

For the present investigation, an SBF (“simulated body fluid”) was used which consists of an aqueous solution of the following salts:

| | | | |
|---|---|---|---|
| Na⁺ | 14 mM | PO₄³⁻ | 4.7 mM |
| K⁺ | 21 mM | Cl⁻ | 30 mM |
| Ca²⁺ | 1.8 mM | | |

TABLE 4
Composition of the SBF solution (1 l).

| Chemical | Actual [g] | |
|---|---|---|
| CaCl₂ × 2H₂O | 1.1026 | g |
| KCl | 48.4574 | g |
| KH₂PO₄ | 0.6129 | g |
| HEPES | 23.8298 | g |
| NaOH (2 M) | 13.62 | g |

100 μl of a formulation of 1.2% by weight of apatite as a powder or in the form of the apatite-gelatin composite according to 1.1 and a defined amount of fluoride were dissolved or dispersed in water at 37° C., pipeted into 30 ml of the SBF and the subsequent pH change was monitored by means of a pH electrode (Inlab 410, Mettler Toledo; measuring apparatus: Consort, multiparameter analyzer C833). The pH decrease ApH after 3 hours serves as a particularly suitable measure.

### 5.2 Measurement Results.

These pH value changes can be explained by a precipitation of calcium phosphate from the artificial saliva induced by the poorly water-soluble calcium salt or its composites, which is measured according to the following equation, by means of the falling pH in the “SBF”:

10 CaCl₂+6Na₂HPO₄+2H₂O→Ca₁₀(OH)₂(PO₄)₆+12NaCl+8HCl

On the basis of the results, the mode of action of the poorly soluble calcium salts (without wanting to be restricted to this theory) does not only lie in the provision of calcium and/or phosphate ions for incorporation into the dentinal canaliculi and the enamel. The poorly soluble calcium salt is, in particular, able to deposit hydroxyapatite from the calcium and phosphate ions present in supersaturated concentrations in the natural saliva of humans.

Thus, a pH decrease occurring particularly early, or a particularly strong pH decrease in an experiment of this type, shows a particularly high nucleation potential of the composite material used. Advantages of a particularly high nucleation potential are, in particular, a more rapid onset of the action and a more effective neomineralization.

This nucleation action thus leads to a buildup of new dental material (neomineralization), in particular, of enamel and/or of dentine, from the endogenous reservoir.

As is revealed from Table 5, fluoride-containing compositions show a markedly greater pH decrease than fluoride-free compositions. Accordingly, in the presence of fluoride a larger amount of apatite is formed than in the absence of fluoride. Surprisingly, it is possible here for the action of fluoride and apatite to be combined. Fluoride on its own does not lead to any comparable apatite formation, which is why it is possible to speak of a synergistic behavior. The effect is likewise stable over time and even after 84 days (84 d) can still be observed almost unchanged.

TABLE 5

| | ΔpH | ΔpH | ΔpH | ΔpH | ΔpH |
|---|---|---|---|---|---|
| | 3 h | 1 d | 7 d | 14 d | 84 d |
| Fluoride | 0.18 | — | — | — | — |
| Apatite-protein | 0.46 | — | — | — | — |
| composite | | | | | |
| Apatite-protein | 1.03 | 0.90 | 1.00 | 0.86 | 0.87 |
| composite + fluoride | | | | | |

The remineralizing action of a formulation is customarily also investigated by the method of microradiography (Lagerweij M D, Damen J J, ten Cate J M Caries Res 1996a; 30:231-236). This method measures the mineral content of teeth by means of the X-ray density of specially prepared enamel platelets. Since the platelets are X-rayed in cross-section, it is possible to prepare a depth profile of the mineral content and to determine the total loss of mineral by means of the integral.

In the experiment, 5 platelets deionized beforehand were each treated with one of the following formulations:

carboxymethylcellulose (CMC) gel containing 900 ppm of fluoride

carboxymethylcellulose gel containing apatite-protein composite (1%, according to 1.1)+900 ppm of fluoride.

Treatment was carried out twice daily and extended over 4 weeks. Of this, treatment was initially carried out for two weeks under remineralizing conditions (pH 7) and subsequently for two weeks under demineralizing conditions (pH 5), in approximation to the above-mentioned reference. In each case, microradiographic investigations were carried out. A suitable parameter which images the deep-mineralizing properties is the integrated mineral loss (IML).

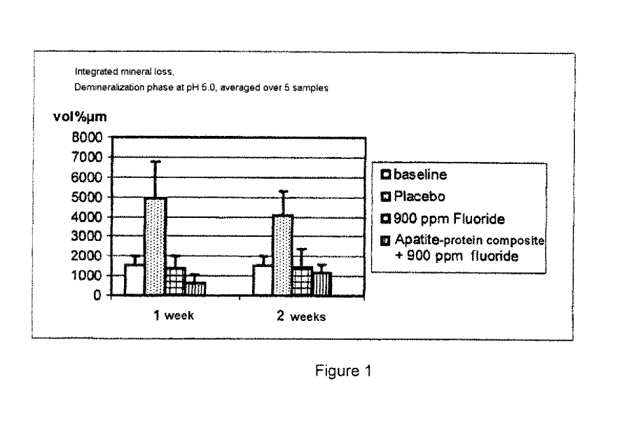

The results shown in FIG. 1 make it clear that fluoride markedly suppresses the mineral loss. A use according to the invention of apatite-protein composite containing fluoride does not lead to any worsening of the mineral loss, but to an improvement in the action. In formulations according to the prior art, the fluoride is lost by absorption on the apatite and leads to an increase in the mineral loss.

### 7. Hardening of the Neomineralized Layer by Fluoride

AFM (atomic force microscope) nanoindents are a recognized method for the determination of the hardness of dental material (M. Finke et al., Surface Science 2001, 491, 456-467). The advantage compared to the measurement of the microhardness lies in the possibility that the hardness of very thin layers can also be determined without artifacts of the underlying material.

In the experiment, 20 nanoindents each were carried out on differently treated cattle dentine disks and the values of the penetration depth obtained were averaged.

The following formulations were employed for the treatment of the dentine disks (see Example 6)

apatite-protein composite (1%)+1500 ppm of fluoride Formulation 1

apatite-protein composite (1%) Formulation 2

Treatment was carried out 2 times daily and lasted 10 days. Between the treatments, the dentine disks were stored in the SBF described. Before the hardness measurement, it was ensured electron microscopically by means of the closure of the dentinal canaliculi that a neomineralized layer had been formed.

TABLE 6

| Dentine disk treated with | Penetration depth (nm) |
|---|---|
| Formulation 1 | 45.6 ± 5.9 |
| Formulation 2 | 88.1 ± 8.7 |
| Untreated | 62.1 ± 4.6 |

The deeper the cantilever of the AFM penetrates into the surface, the softer the material. From the results in Table 6, it is clear that the presence of fluoride also hardens the newly formed, neomineralized material. This means that fluoride synergistically increases the action of the composite, as no neomineralization can be observed in compositions containing fluoride alone. This effect protects the tooth from acid attack and counteracts the erosion of the dentine.

### Example 9

### Microhardness of Neomineralized Layers and Erosion Stability

Microhardness is a method recognized in dental research for the assessment of mineralization states of teeth in vitro (Meurman et al., Scand. J. Dent. Res. 1990 (98) 568-570). If the enamel of the tooth surface is deionized by damaging exogenous influences (erosion by acidic drinks or lesion formation due to metabolic products of bacteria), a markedly decreased microhardness is found (softened enamel).

For the measurement of the microhardness according to Knoop, pressure is exerted with a defined weight force on the enamel surface using a ground diamond in the form of a rhombic pyramid and the length of the long diagonal of the impression is measured. The hardness index can then be calculated according to a defined formula.

Study Design.

The enamel disks used for carrying out the study are prepared from front teeth of cattle. The preparations have dimensions of about 5×5 mm². These are embedded in acrylic resin. The surface of the sample bodies is polished.

Two groups of 5 enamel disks each are investigated. Before the start of the study, the hardness of the untreated samples is measured. The samples of groups 1 and 2 are deionized at 37° C. for 6 h in a lactic acid buffer (0.1 M, pH=4.6). Subsequently, the samples are carefully rinsed with completely deionized water and dried. The micro-hardness of these groups is measured.

Stage 1: Remineralization.

The samples of group 1 are treated with a toothgel according to the invention (according to Table 2, 4.1) and the samples of group 2 with a toothgel according to the prior art containing 1,500 ppm of fluoride (without calcium salt to be used according to the invention and/or composite material). The treatment is carried out using an automated immersion apparatus according to the following scheme.

The samples were fixed to a sample carrier using a medical thermoplastic and immersed alternately in a toothpaste suspension (2 parts of water:1 part of toothpaste) for 5 min and an SBF solution (25 min) (for formulation see Table 3). Every 2 h, the SBF solution was replaced. This treatment was carried out between 0800 and 1800. In the remaining period of the day, the treatment with SBF solution was prolonged to 55 min. The toothpaste suspension was changed once per day. This treatment was carried out for 5 days. After the end of the treatment, the samples were carefully rinsed with completely deionized water, dried and analyzed by means of the microhardness.

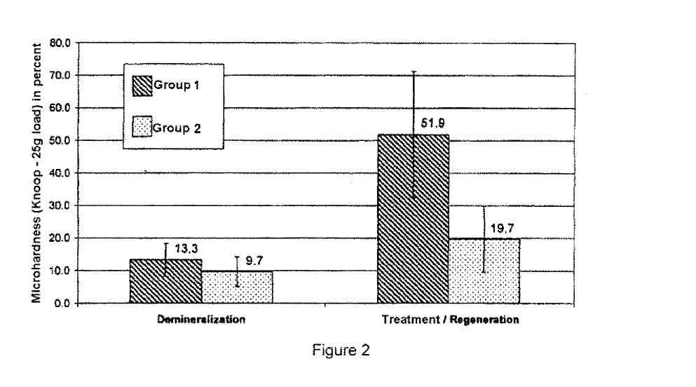

It is seen that the toothpaste according to the invention leads to a markedly better recovery of the tooth hardness than is observed for a composition according to the prior art. (FIG. 2).

Stage 2: Erosion.

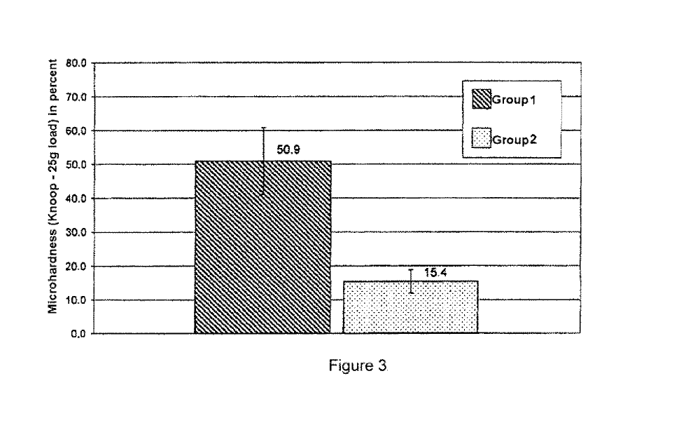

The samples of groups 1 and 2 were subsequently immersed in a cola lemonade for 5 min (pH=2.5). After this treatment, the samples were carefully rinsed with completely deionized water and dried. Subsequently, the microhardness of the samples was determined. In the group which was treated with toothpaste according to the prior art, the exposure to acid-containing lemonade leads to additional, severe damage, as the further decrease in hardness shows. The group which was treated with toothpaste according to the invention remains, on the other hand, at the hardness level as before exposure in relation to the acid. The effect observed consequently leads to protection from erosion and caries. (FIG. 3).

### Example 8

In order to examine the action of the embodiment according to the invention, enamel platelets from front teeth of cattle were prepared, embedded in acrylic resin and polished. A toothpaste according to the invention was tested against a toothpaste according to the prior art. Ten samples were employed per group.

In order to attain clinically relevant conditions which were as close to reality as possible, the samples were treated in a cycling model which exposes the samples to acid stress exceeding normal nutrition habits.

The model has the following course and follows published models (ten Cate et al., Eur. J. Oral Sci., 1995: 103; 362-367).
1) immersion for 5 minutes in a suspension of the respective toothpaste (1 part of toothpaste, 2 parts of water)
2) storage for 25 minutes in synthetic saliva (composition according to Table 3) (37° C.)
3) repetition of 1) and 2) 4 times
4) acid stress for 30 minutes (lactic acid, pH 4.6)
5) repetition of 1)-4) 4 times
6) carrying out of 1)-5) for 10 days.

During the night, the samples were stored under 100% atmospheric humidity at room temperature.

After the treatment, the samples were divided and polished on the cut edge. Following this, electron microscopic investigation was carried out in plan and in cross-section.

The enamel samples treated with the toothpaste according to the invention show the formation of a layer 4-6 μm thick (FIG. 4), which is intimately connected to the natural enamel. In samples which were treated with toothpastes according to the prior art, the effect is not seen.

FIGS. 4 a and b are two representative electron micrographs of a cross-section of an enamel sample treated with toothpaste according to the invention. A layer sealing the natural enamel is clearly to be discerned (In FIG. 4 a, white arrows mark the boundary of the layer to the natural enamel). In the contrasted photograph of FIG. 4 b), the ordered structure is clear.

Table 7 shows comparative elemental analyses in the area of the layer and in the area of the underlying natural enamel. The data show that the phosphorus and calcium content protect the natural enamel of the tooth.

### Table 7

Comparison of the calcium/phosphorus ratios of the layers produced by the toothpaste according to the invention compared with values of the natural enamel (averaged over 5 measurements).

| Sample location | Ca/P wt. | Ca/P at. |
|---|---|---|
| Resulting layer | 2.11 | 1.63 |
| Natural enamel | 2.05 | 1.58 |
| Apatite (theoretical) | 2.15 | 1.66 |

wt.: weight ratio
at.: atomic ratio

1. A method for the remineralization and/or neomineralization of a tooth comprising contacting a tooth with an effective amount of a composition comprising a composite comprising at least one poorly water-soluble calcium salt and a polymer selected from the group consisting of a protein, a polyelectrolyte and a polysaccharide.

2. The method of claim 1 wherein the poorly water-soluble calcium salt contained is selected from the group consisting of hydroxyapatite and fluoroapatite.

3. The method of claim 2 wherein the poorly water-soluble calcium salt comprises hydroxyl and/or carbonate groups.

4. The method of claim 1 wherein the poorly water-soluble calcium salt is present in the form of individual crystallites or in the form of particles comprising a plurality of said crystallites wherein the mean diameter of the particles is below 1,000 nm.

5. The method of claim 4 wherein the particles are rod-shaped and/or lamellar.

6. The method of claim 5 wherein the particles are lamellar having a length of from 10 to 150 nm and a breadth of from 5 to 150 nm.

7. The method of claim 5 wherein the particles have a ratio of length to breadth of from 1 to 4.

8. The method of claim 4 wherein the particles have an area of 0.1×10⁻¹⁵ m² to 90×10⁻¹⁵ m².

9. The method of claim 4 wherein the individual crystallite has a thickness in the range from 2 to 50 nm and a length in the range from 10 to 150 nm.

10. The method of claim 1 wherein the amount of the polymer component is from 0.1 to 80% by weight of the total weight of the composite material.

11. The method of claim 1 wherein the polyelectrolyte is selected from the group consisting of polyaspartic acids, alginic acids, pectins, deoxyribonucleic acids, ribonucleic acids, polyacrylic acids and polymethacrylic acids.

12. The method of claim 1 wherein the polysaccharide is selected from the group consisting of glucuronic acid and iduronic acid-containing polysaccharides.

13. The method of claim 12 wherein the polysaccharide is selected from chondroitin sulfate, heparin, hyaluronic acid, and xanthan.

14. The method of claim 1 wherein the protein is selected from protein hydrolyzates and protein hydrolyzate derivatives.

15. The method of claim 1 wherein the protein component mentioned is selected from the group consisting of collagen, gelatin, keratin, casein, wheat protein, rice protein, soybean protein, almond protein, hydrolyzates thereof and hydrolyzate derivatives thereof.

16. The method of claim 15 wherein the protein is selected from the group consisting of gelatin, casein and hydrolyzates thereof.

17. The method of claim 1 wherein the crystallites or particles of the calcium salts are covered by one or more surface modification agents.

18. The method of claim 1 wherein the composition is further comprised of fluoride.
